(19) United States
(12) Patent Application Publication
(10) Pub. No.: US 2015/0125514 A1
(43) Pub. Date: May 7, 2015

(54) BIOMARKER

(71) Applicants: Socrates Tzartos, Kaisariani (GR); Ioannis Tzartos, Kaisariani (GR); Christos Stergiou, Ilion (GR); Konstantinos Killindreas, Kalamiki (GR)

(72) Inventors: Socrates Tzartos, Kaisariani (GR); Ioannis Tzartos, Kaisariani (GR); Christos Stergiou, Ilion (GR); Konstantinos Killindreas, Kalamiki (GR)

(21) Appl. No.: 14/352,562

(22) PCT Filed: Aug. 14, 2012
(86) PCT No.: PCT/IB2012/054142

§ 371 Date: Apr. 17, 2014

(30) Foreign Application Priority Data
Oct. 17, 2011 (GR) 10110100593

(51) Int. Cl. G01N 33/564; C07K 16/28; A61K 39/00; C07K 14/705

(52) U.S. Cl. CPC G01N 33/564; C07K 14/705; C07K 16/28; A61K 39/0008; G01N 2333/705; G01N 2800/52; A61K 2039/577; G01N 2500/02
USPC 424/450; 435/7.1; 435/7.21; 435/7.92; 436/501; 424/185.1; 424/139.1; 210/691; 210/679

(57) ABSTRACT

A method of diagnosis, prognosis or treatment of an autoimmune disease in a subject wherein the method comprises detecting aquaporin-1 (AQP1), aquaporin-2 (AQP2), aquaporin-5 (AQP5), aquaporin-7 (AQP7) and/or aquaporin-8 (AQP8) autoantibodies in a sample obtained from said subject.

### FIELD OF THE INVENTION

This invention relates to novel autoantibody markers against aquaporin-1 (AQP1), aquaporin-2 (AQP2), aquaporin-5 (AQP5), aquaporin-7 (AQP7) and aquaporin-8 (AQP8) and methods of diagnosing and treating diseases, in particular diseases of the nervous system and autoimmune diseases.

### BACKGROUND OF THE INVENTION

The use of biomarkers in clinical diagnostics and drug development is becoming increasingly important. However, the identification of relevant biomarkers often represents a significant challenge. For example, in most instances, no specific biomarkers for lesions or mechanisms of lesion formation in the central nervous system (CNS) are known, with viruses or autoimmunity against unknown antigens being proposed.

Aquaporins are a group of channel proteins, important for the rapid transport, primarily of water, in response to osmosis (Pasantes-Morales and Cruz-Rangel (2010) Neuroscience 168(4): 871-884). These proteins play a role in water balance in the CNS crucial for the integrity of the CNS parenchyma (Amity-Moghaddam and Ottersen (2003) Nat Rev Neurosci 4(12): 991-1001; Lukaszewicz et al. (2011) Curr Opin Anaesthesiol 24(2): 138-143).

Aquaporin 4 (AQP4) and aquaporin 1 (AQP1) are the main aquaporins which have been detected in the brain (Badaut et al. (2002) J Cereb Blood Flow Metab 22(4): 367-378; Satoh et al. (2007) Neuropathology 27(3): 245-256). In mice, the absence of AQP4 prevents cytotoxic edema during ischemia (Manley et al. (2000) Nat Med 6(2): 159-163; Amiry-Moghaddam and Ottersen (2003) Nat Rev Neurosci 4(12): 991-1001), but inhibits the resolution of vasogenic edema (Papadopoulos et al. (2004) FASEB J 18(11): 1291-1293).

AQP1 in mice is not expressed in astrocytes but is detected specifically in the epithelium of the choroid plexus and in some neurons and may be involved in cerebrospinal fluid (CSF) formation without a direct role in brain edema. In an ischemic mouse brain model, no effect of AQP1 levels or cell types expressing AQP1 was detected during brain ischemia (Ribeiro Mde et al. (2006) J Neurosci Res 83(7): 1231-1240).

In addition to the CNS, AQP1 has been also detected in the peripheral nervous system (Ma, T. H., H. W. Gao, et al. (2011) Acta Pharmacol Sin 32(6): 711-715) and in non-neuronal tissues like red blood cells, liver, kidney, lungs, and sweat glands.

The presence of autoantibodies against mouse astrocytic end feet has been selectively detected in sera from patients with demyelinated lesions in the CNS, usually with the specific characteristics of neuromyelitis optica, NMO (Lennon et al. (2004) Lancet 364(9451): 2106-2112). NMO is a chronic inflammatory demyelinating disease of the CNS, mainly affecting the optic nerves and the spinal cord. Like multiple sclerosis (MS), NMO usually follows a relapsing-remitting course, but it often leads to more severe disability with impairment of functional vision and/or loss of ambulation. Its differentiation from MS and other similar CNS diseases is important in order to apply the appropriate treatment. However, such differentiation is usually very difficult due to overlapping clinical presentation and magnetic resonance imaging (MRI) findings.

The autoantibodies against astrocytic end feet have been called NMO-IgG. Furthermore, it was shown that NMO-IgG bind selectively to the AQP4 water channel (Lennon et al. (2005) J Exp Med 202(4): 473-477), linking NMO with regional loss of AQP4 from astrocytes (Kale et al. (2009) Arch Neurol 66(10): 1298-1299). In vitro, the pathogenic role of anti-AQP4 antibodies has been shown, with accumulations of glutamate, possibly causing a pathogenic environment to oligodendrocytes and neurons (Hinson et al. J Exp Med 205(11): 2473-2481).

Several other AQPs have been detected in various regions and cell types of mammalian CNS (mostly studied in rodents) including astrocytes, oligodendrocytes, neurons and ependymal cells. More specifically,
a. Aquaporin-2 (AQP2) has been detected in several regions of the CNS including the ependymal cell layer, spinal cord, subcortical white matter and hippocampus (Mobasheri et al., 2005, J. Mol. Histol. 36, 1-14).
b. Aquaporin-5 (AQP5) has been detected in astrocytes and neurons in culture (Yamamoto et al., 2001, Brain Res. Mol. Brain Res. 90, 26-38). Its expression showed a transient up-regulation and subsequent down-regulation within 20 h of reoxygenation after hypoxia. This suggests that AQP5 may contribute to the induction of the intracranial edema in the CNS after ischemia injury (Albertini and Bianchi, 2010, Curr. Neuropharmacol. 2010 (8), 84-91).
c. Aquaporin-7 (AQP7) was found in choroid plexus, ependyma, pia and blood vessels of the mouse brain during perinatal development (Shin et al. 2006, Neurosci. Lett. 409, 106-111).
d. Aquaporin-8 (AQP8) expression was found in the spinal cord, especially in the ependymal cells lining its central canal, and possibly in astrocytes (Oshio et al. 2004, Neuroscience, 127, 685-693). Based on these findings it was suggested that AQP8 may facilitate water transport into the central canal of the spinal cord (Albertini and Bianchi, 2010, Curr. Neuropharmacol. 2010 (8), 84-91).

In addition to their presence and function in the brain, these AQPs are also present in other organs and tissues of the body (reviewed in Verkman 2011, J Cell Sci. 2011 124:2107-12; Verkman 2012 Annu Rev Med. 63:303-16). Specifically,

AQP2 is the only AQP regulated by vasopressin expressed in kidney cells for reabsorption of water. In humans, genetic mutations in AQP2 result in nephrogenic diabetes insipidus, characterized by polyuria and urinary hypo-osmolality.

AQP5, is expressed in the kidney, salivary and airway submucosal glands, show defective secretion of saliva and airway mucus and tears.

AQP7 is expressed in adipocytes and facilitates glycerol in order to prevent fat accumulation.

AQP8 is expressed in organs of the mammalian gastrointestinal tract (salivary gland, liver, pancreas, small intestine, and colon) and in the testes, heart, kidney, and airways.

The present invention identifies anti-AQP1, aquaporin-2 (AQP2), aquaporin-5 (AQP5), aquaporin-7 (AQP7) and aquaporin-8 (AQP8) antibodies as a novel biomarkers for disease.

### SUMMARY OF THE INVENTION

According to a first aspect of the invention there is provided a method of diagnosis or prognosis of an autoimmune disease in a subject wherein the method comprises detecting antibodies to aquaporin-1 (herein referred to as AQP1 autoantibodies), aquaporin-2 (herein referred to as AQP2 autoantibodies), aquaporin-5 (herein referred to as AQP5 autoantibodies), aquaporin-7 (herein referred to as AQP7 autoantibodies) and/or aquaporin-8 (herein referred to as AQP8 autoantibodies) in a sample obtained from said subject.

The AQP1, AQP2, AQP5, AQP7 and/or AQP8 autoantibodies may be detected in the same or different samples obtained from said subject.

In a preferred embodiment the method comprises detecting AQP1 autoantibodies.

The method according to this aspect of the invention may further comprise detecting aquaporin-4 (AQP4) autoantibodies in the same or a different sample obtained from said subject.

In one embodiment, the level of AQP1, AQP2, AQP5, AQP7 and/or AQP8 autoantibodies is compared to the corresponding level of AQP1, AQP2, AQP5, AQP7 and/or AQP8 autoantibodies in a sample from a healthy subject. By a healthy subject it is meant a corresponding subject that does not have an autoimmune disease characterised by AQP1, AQP2, AQP5, AQP7 and/or AQP8 autoantibody expression.

According to a second aspect of the present invention there is provided a method of monitoring the progress of an autoimmune disease in a subject comprising the steps of:
(i) providing a first sample from the subject,
(ii) detecting AQP1, AQP2, AQP5, AQP7 and/or AQP8 autoantibodies in said first sample,
(iii) providing a second or subsequent sample from the subject wherein said second sample is obtained from the subject after said first sample,
(iv) comparing the level of the AQP1, AQP2, AQP5, AQP7 and/or AQP8 autoantibodies in the second sample with the corresponding levels of AQP1, AQP2, AQP5, AQP7 and/or AQP8 autoantibodies in the first sample.

The AQP1, AQP2, AQP5, AQP7 and/or AQP8 autoantibodies may be detected in the same or different first sample obtained from said subject and the same or different second or subsequent sample obtained from said subject.

An increase in AQP1, AQP2, AQP5, AQP7 and/or AQP8 autoantibodies expression in the second sample relative to the first sample may indicate disease progression. A decrease in AQP1, AQP2, AQP5, AQP7 and/or AQP8 autoantibodies expression in the second sample relative to the first sample may indicate disease regression.

In a preferred embodiment the method comprises monitoring the progress of an autoimmune disease in a subject comprising the steps of:
(i) providing a first sample from the subject,
(ii) detecting AQP1 autoantibodies in said first sample,
(iii) providing a second or subsequent sample from the subject wherein said second sample is obtained from the subject after said first sample,
(iv) comparing the level of the AQP1 autoantibodies in the second sample with the level of AQP1 autoantibodies in the first sample.

The method according to this aspect of the invention may also involve detecting AQP4 autoantibodies in said first and second or subsequent samples.

The AQP4 autoantibodies may be detected in the same or different first sample obtained from said subject and the same or different second or subsequent sample obtained from said subject.

Preferably the second sample is obtained from the subject at least 1, 2, 5, 10, 20, 50, 100, 200 or 300 days after the first sample.

The second aspect of the invention may be used to monitor the progress of an autoimmune disease in response to treatment e.g. immunomodulatory therapy (immunosuppression, plasmapheresis, Ig-apheresis, IVIG, therapeutic administration of AQP1, AQP2, AQP5, AQP7 and/or AQP8 in various forms including in liposomes or nanoparticles, or by nasal or oral administration, T-cell therapy etc). Thus, the present invention may be used to assess the effectiveness of a treatment. The treatment may be administered between the taking of the first sample and the taking of the second or a subsequent sample from the patient.

The AQP1, AQP2, AQP5, AQP7 and/or AQP8 autoantibodies may be detected by contacting said sample with the respective AQP1, AQP2, AQP5, AQP7 and/or AQP8 protein or fragment thereof, or a cell or tissue comprising said polypeptide or fragment, and detecting the presence or absence of binding of said AQP1, AQP2, AQP5, AQP7 and/or AQP8 autoantibodies to said polypeptide, fragment, cell or tissue.

Thus, the AQP1 protein or fragment, or cell or tissue comprising the same is used to bind AQP1 autoantibodies, the AQP2 protein or fragment, or cell or tissue comprising the same is used to bind AQP2 autoantibodies, the AQP5 protein or fragment, or cell or tissue comprising the same is used to bind AQP5 autoantibodies, the AQP7 protein or fragment, or cell or tissue comprising the same is used to bind AQP7 autoantibodies, and the AQP8 protein or fragment, or cell or tissue comprising the same is used to bind AQP8 autoantibodies.

In a preferred embodiment, the AQP1 autoantibodies are detected by contacting said sample with an AQP1 protein or fragment thereof, or a cell or tissue comprising said polypeptide or fragment, and the presence or absence of binding of said AQP1 autoantibodies to said polypeptide, fragment, cell or tissue is detected.

According to another aspect of the present invention there is provided a method of detecting the presence or absence of AQP1, AQP2, AQP5, AQP7 and/or AQP8 autoantibodies in a sample obtained from a subject comprising contacting said sample with the respective AQP1, AQP2, AQP5, AQP7 and/or AQP8 protein or fragment thereof, or a cell or tissue comprising said polypeptide or fragment, and detecting the presence or absence of binding of said AQP1, AQP2, AQP5, AQP7 and/or AQP8 autoantibodies to said polypeptide, fragment, cell or tissue. (e.g. human embryonic kidney (HEK) cells. mouse brain sections, liver sections or any other AQP1, AQP2, AQP5, AQP7 and/or AQP8-containing tissue). The AQP1, AQP2, AQP5, AQP7 and/or AQP8 autoantibodies may be detected in the same or different samples obtained from said subject.

In a preferred embodiment the method comprises detecting the presence or absence of AQP1 autoantibodies in a sample obtained from a subject comprising contacting said sample with an AQP1 protein or fragment thereof, or a cell or tissue comprising said polypeptide or fragment, and detecting the presence or absence of binding of said AQP1 autoantibodies to said polypeptide, fragment, cell or tissue. (e.g. human embryonic kidney (HEK) cells, mouse brain sections, liver sections or any other AQP1-containing tissue).

The presence of binding of said AQP1, AQP2, AQP5, AQP7 and/or AQP8 polypeptide, fragment, cell or tissue to said AQP1, AQP2, AQP5, AQP7 and/or AQP8 autoantibodies may be indicative of an autoimmune disease in said individual.

The sample used in the present invention may be, but is not limited to, tissue biopsy, nervous tissue, blood, blood cells, blood plasma, blood serum, blood immunoglobulins or cerebrospinal fluid.

Preferably the detection of AQP1, AQP2, AQP5, AQP7 and/or AQP8 autoantibodies described herein is achieved using an immunoassay such as an enzyme-linked immunosorbent assay (ELISA), radioimmunoassay (RIA), immunoprecipitation assay, immunofluorescence assay (IFA), indirect immunofluorescence assay (IIF), enzyme linked assay (EIA), luminescence immunoassay (LIA), Western Blot assay (WB) or cell based assay (CBA).

According to another aspect of the present invention there is provided an AQP1, AQP2, AQP5, AQP7 and/or AQP8 polypeptide or a fragment thereof, or a cell or tissue comprising said cell or tissue, for use in diagnosing or determining the progression of a disease of the CNS.

In a preferred embodiment there is provided an AQP1 polypeptide or a fragment thereof, or a cell or tissue comprising said cell or tissue, for use in diagnosing or determining the progression of a disease of the CNS.

Preferably, the autoimmune disease referred to herein is an autoimmune disease of the CNS.

Preferably, the autoimmune disease referred to herein is a disease associated with the formation of demyelinated lesions. Demyelination is destruction/loss of myelin sheath from a myelinated area of the nervous system. The autoimmune disease referred to herein may be a Neuromyelitis Optica (NMO) spectrum disorder or multiple sclerosis. The NMO spectrum disorder may be:
1. NMO;
2. a limited form of NMO such as:
   a. Longitudinally extensive tranverse myelitis
   b. Optic neuritis, recurrent or simultaneous bilateral;
3. Asian Optic-Spinal Multiple Sclerosis;
4. Optic Neuritis or Longitudinally Extensive Myelitis Associated With Systemic Autoimmune Disease; or
5. Optic Neuritis or Myelitis Associated With Neuromyelitis Optica-Typical Brain Lesions (from Wingerchuk D M, Lennon V A, Lucchinetti C F, et al. The spectrum of neuromyelitis optica. Lancet Neurol 2007; 6(9):805-815.)

The methods according to the present invention may relate to the diagnosis, treatment, monitoring and prognosis of one or more autoimmune diseases.

Autoimmune disease referred to herein may be characterised by the presence of, or elevated levels of, AQP1, AQP2, AQP5, AQP7 and/or AQP8 autoantibody.

According to another aspect of the present invention there is provided a method of treating a subject comprising withdrawing a body fluid containing AQP1, AQP2, AQP5, AQP7 and/or AQP8 autoantibodies; removing a substantial portion of the autoantibodies from the body fluid; and returning the body fluid to the subject.

In a preferred embodiment the method comprises withdrawing a body fluid containing AQP1 autoantibodies; removing a substantial portion of the autoantibodies from the body fluid; and returning the body fluid to the subject.

The method of treatment may comprise removing a substantial portion of AQP1, AQP2, AQP5, AQP7 and/or AQP8 autoantibodies as well as AQP4 autoantibodies.

According to another aspect of the present invention there is provided use of an AQP1, AQP2, AQP5, AQP7 and/or AQP8 polypeptide or a fragment thereof, or a cell or tissue comprising said polypeptide or a fragment, in an ex-vivo method of immunoadsorption of AQP1, AQP2, AQP5, AQP7 and/or AQP8 autoantibodies.

In a preferred embodiment there is provided use of an AQP1 polypeptide or a fragment thereof, or a cell or tissue comprising said polypeptide or a fragment, in an ex-vivo method of immunoadsorption of AQP1 autoantibodies.

According to another aspect of the present invention there is provided a solid support for use in a method of immunoadsorption of AQP1, AQP2, AQP5, AQP7 and/or AQP8 autoantibodies comprising an AQP1, AQP2, AQP5, AQP7 and/or AQP8 polypeptide or fragment thereof, or a cell or tissue comprising said polypeptide or a fragment, wherein said polypeptide, fragment, cell or tissue is bound to the solid support.

In a preferred embodiment there is provided a solid support for use in a method of immunoadsorption of AQP1 autoantibodies comprising an AQP1 polypeptide or fragment thereof, or a cell or tissue comprising said polypeptide or a fragment, wherein said polypeptide, fragment, cell or tissue is bound to the solid support.

The solid support may be, for example, a membrane, a bead, porous glass, silica, a resin or a synthetic matrix.

According to another aspect of the present invention there is provided the use of a solid support of the invention in an ex-vivo method of immunoadsorption of AQP1, AQP2, AQP5, AQP7 and/or AQP8 autoantibodies.

In a preferred embodiment there is provided the use of a solid support of the invention in an ex-vivo method of immunoadsorption of AQP1 autoantibodies.

According to another aspect of the present invention there is provided a method of identifying compounds capable of alleviating or treating disease characterised by elevated levels of AQP1, AQP2, AQP5, AQP7 and/or AQP8 autoantibody, the method comprising: contacting a candidate compound in the presence of AQP1, AQP2, AQP5, AQP7 and/or AQP8 polypeptide or fragment thereof and an antibody capable of specifically binding AQP1, AQP2, AQP5, AQP7 and/or AQP8, wherein a compound that prevents binding of the antibody to AQP1, AQP2, AQP5, AQP7 and/or AQP8 or a fragment thereof is a candidate for treating an autoimmune disorder.

In a preferred embodiment there is provided a method of identifying compounds capable of alleviating or treating disease characterised by elevated levels of AQP1 autoantibody, the method comprising: contacting a candidate compound in the presence of AQP1 polypeptide or fragment thereof and an antibody capable of specifically binding AQP1, wherein a compound that prevents binding of the antibody to AQP1 or a fragment thereof is a candidate for treating an autoimmune disorder.

According to another aspect of the present invention there is provided an animal model for use in screening for compounds that treat or prevent a disease characterised by elevated levels of AQP1, AQP2, AQP5, AQP7 and/or AQP8 autoantibodies wherein said animal model expresses or contains AQP1, AQP2, AQP5, AQP7 and/or AQP8 autoantibodies.

In a preferred embodiment there is provided an animal model for use in screening for compounds that treat or prevent a disease characterised by elevated levels of AQP1 autoantibodies wherein said animal model expresses or contains AQP1 autoantibodies.

Preferably the animal model is a mouse, rat, guinea pig, rabbit or non-human primate animal model.

In one embodiment, the animal is injected with human anti-AQP1, AQP2, AQP5, AQP7 and/or AQP8 antibodies. In another embodiment the animal is immunized with the AQP1, AQP2, AQP5, AQP7 and/or AQP8 or fragment thereof.

According to another aspect of the present invention there is provided a method of screening for an agent that modulates the binding of an AQP1, AQP2, AQP5, AQP7 and/or AQP8 antibody to the respective AQP1, AQP2, AQP5, AQP7 and/or AQP8 polypeptide or fragment thereof comprising:
(i) contacting an AQP1, AQP2, AQP5, AQP7 and/or AQP8 polypeptide or fragment thereof with the respective AQP1, AQP2, AQP5, AQP7 and/or AQP8 antibody in the presence and absence of said agent; and
(ii) determining whether binding of AQP1, AQP2, AQP5, AQP7 and/or AQP8 polypeptide or fragment thereof to the respective AQP1, AQP2, AQP5, AQP7 and/or AQP8 antibody is modulated in the presence of said agent.

In a preferred embodiment there is provided a method of screening for an agent that modulates the binding of an AQP1 antibody to an AQP1 polypeptide or fragment thereof comprising:
(i) contacting an AQP1 polypeptide or fragment thereof with the AQP1 antibody in the presence and absence of said agent; and
(ii) determining whether binding of AQP1 polypeptide or fragment thereof to the AQP1 antibody is modulated in the presence of said agent.

According to another aspect there is provided an AQP1, AQP2, AQP5, AQP7 and/or AQP8 polypeptide or fragment thereof for use in treating a disease characterised by the presence of, or elevated levels of, AQP1, AQP2, AQP5, AQP7 and/or AQP8 autoantibody wherein the AQP1, AQP2, AQP5, AQP7 and/or AQP8 polypeptide or fragment reduces or eliminates the respective anti-AQP1, AQP2, AQP5, AQP7 and/or AQP8 antibodies from the circulation. The AQP1, AQP2, AQP5, AQP7 and/or AQP8 polypeptide or fragment thereof may be incorporated into a particle such as a nanoparticle or a liposome.

In a preferred embodiment there is provided an AQP1 polypeptide or fragment thereof for use in treating a disease characterised by the presence of, or elevated levels of, AQP1 autoantibody wherein the AQP1 polypeptide or fragment reduces or eliminates anti-AQP1 antibodies from the circulation.

According to another aspect there is provided an AQP1, AQP2, AQP5, AQP7 and/or AQP8 polypeptide or fragment thereof for use in immunotherapy for treating a disease characterised by the presence of, or elevated levels of, AQP1, AQP2, AQP5, AQP7 and/or AQP8 autoantibody wherein the AQP1, AQP2, AQP5, AQP7 and/or AQP8 polypeptide or fragment thereof modulates the immune response against AQP1, AQP2, AQP5, AQP7 and/or AQP8 in the subject. The AQP1, AQP2, AQP5, AQP7 and/or AQP8 polypeptide or fragment thereof may be incorporated into a particle such as a nanoparticle or a liposome.

In a preferred embodiment there is provided an AQP1 polypeptide or fragment thereof for use in immunotherapy for treating a disease characterised by the presence of, or elevated levels of, AQP1, autoantibody wherein the AQP1 polypeptide or fragment thereof modulates the immune response against AQP1 in the subject.

According to another aspect of the invention there is provided a method of treating a subject having a disease characterised by the presence of, or elevated levels of, AQP1, AQP2, AQP5, AQP7 and/or AQP8 autoantibody comprising administration to said subject an agent which binds to the subject's AQP1, AQP2, AQP5, AQP7 and/or AQP8 and protects it against the pathogenic AQP1, AQP2, AQP5, AQP7 and/or AQP8 autoantibodies. In one embodiment, the agent may be a non-pathogenic antibody fragment.

In a preferred embodiment there is provided a method of treating a subject having a disease characterised by the presence of, or elevated levels of, AQP1 autoantibody comprising administration to said subject an agent which binds to the subject's AQP1 and protects it against the pathogenic AQP1 autoantibodies.

According to another aspect of the present invention there is provided the use of AQP1, AQP2, AQP5, AQP7 and/or AQP8 polypeptide of fragment thereof for screening for an agent that modulates the interaction between an AQP1, AQP2, AQP5, AQP7 and/or AQP8 polypeptide or fragment thereof and an AQP1, AQP2, AQP5, AQP7 and/or AQP8 antibody.

In a preferred embodiment there is provided the use of AQP1 polypeptide of fragment thereof for screening for an agent that modulates the interaction between an AQP1 polypeptide or fragment thereof and an AQP1 antibody.

According to another aspect of the present invention there is provided the use of AQP1, AQP2, AQP5, AQP7 and/or AQP8 antibody for screening for an agent that modulates the interaction between an AQP1, AQP2, AQP5, AQP7 and/or AQP8 polypeptide or fragment thereof and an AQP1, AQP2, AQP5, AQP7 and/or AQP8 antibody.

In a preferred embodiment there is provided the use of AQP1 antibody for screening for an agent that modulates the interaction between an AQP1 polypeptide or fragment thereof and an AQP1 antibody.

Anti-AQP2, AQP5, AQP7 and/or AQP8 antibodies may act as paraneoplastic markers.

According to another aspect of the present invention there is provided a method of diagnosis or prognosis of cancer in a subject wherein the method comprises detecting aquaporin-1 (AQP1), aquaporin-2 (AQP2), aquaporin-5 (AQP5), aquaporin-7 (AQP7) and/or aquaporin-8 (AQP8) autoantibodies in a sample obtained from said subject.

The AQP1, AQP2, AQP5, AQP7 and/or AQP8 autoantibodies may be detected in the same or different samples obtained from said subject.

In a preferred embodiment there is provided a method of diagnosis or prognosis of cancer in a subject wherein the method comprises detecting aquaporin-1 (AQP1) autoantibodies in a sample obtained from said subject.

The method according to this aspect of the invention may also involve detecting AQP4 autoantibodies in the same or different sample from said subject.

In one embodiment the cancer is nephroma, non-Hodgkin lymphoma or mammary cancer.

### DESCRIPTION OF FIGURES

FIG. 1—Detection of anti-AQP1 antibodies in the sera of patient groups and healthy controls determined by a radioimmunoprecipitation (RIPA) assay

AQP1 was biotinilated and then incubated with ¹²⁵I-streptavidin to result in indirectly radiolabeled AQP1. Precipitated cpm depicts the level of AQP1 bound to the test serum antibodies and indirectly the level of the autoantibodies in the serum. Numbers in parenthesis at the X-axis denote the numbers anti-AQP1 positive versus total tested sera for each group of patients tested, whereas below is the percentage of the positive. The dashed horizontal line denotes the cut-off values for ambiguous and the solid horizontal line denotes the cut-off values for positivity. The x-axis contains, in addition to the precipitated cpm, also the estimated antibody concentration in nM.

FIG. 2. Effect of liver powder treatment of the sera on anti-AQP1 antibody detection

Two anti-AQP1 positive (sera 1 and 2) and two anti-AQP4 positive sera (sera 3 and 4) were pretreated with guinea pig liver powder; their supernatants were tested in RIA with labeled AQP1 and AQP4, respectively, and compared with those of the untreated sera. −, +: serum without or with pretreatment with guinea pig liver powder.

FIG. 3—Plotting precipitated cpm values (corresponding to antibody concentrations) in double-positive sera, for anti-AQP1 versus anti-AQP4 antibodies

Identical RIPAs were performed for the two labeled antigens. Only one serum gave near identical values; 9 sera gave higher values with AQP1 and only 5 sera gave higher values with AQP4.

FIG. 4—Search for cross-reactivity between the anti-AQP1 and anti-AQP4 antibodies

AQP1 and AQP4 were immobilized on ELISA wells. Samples of each test serum were incubated with either immobilized aquaporin, AQP1 (right panel) and AQP4 (left panel), in order to immunoadsorb all antibodies bound to AQP1 or AQP4, respectively. The treated sera were tested by RIPA for binding to labeled AQP1 (2^{nd} bar for each serum) and AQP4 (4^{th} bar for each serum), and compared with the binding of the untreated samples of the same sera (1^{st} and 3^{rd} bars for each serum, respectively).

FIG. 5—Detection of low levels of anti-AQP1 antibodies by the two-step RIPA

AQP1 was immobilized on each ELISA plate well; 150 μl test serum were added per well and incubated. Then the wells were washed, the bound antibodies were eluted by low pH; the pH of the eluted sample was immediately neutralized and the antibody solution was subjected to regular RIPA. Six low positive sera, 4 ambiguous and 2 high background NHS are shown.
Δcpm: the bound cpm by the test serum minus the average of the cpm bound by the NHS sera (256 cpm for the direct RIPA and 330 cpm for the two-step RIPA). Dashed and solid horizontal lines denote the cut offs for ambiguous and positive values.

FIG. 6—Biochemical characterization of the autoantibody-target

A. The sera (a NHS, labeled as N, and three test AQP1+ sera, labeled as 1, 2, 3) were incubated with 0.1 μg biotinilated AQP1 followed by precipitation with Sepharose-protein A beads. The bound AQP1 was released, run on PAGE and revealed by western blot using HRP-streptavidin and its substrate. M, MW standards. B. 0.01 and 0.1 μg of biotinilated AQP1 were directly analised by PAGE followed by Western blot in order to semi-quantitate the intensity of AQP1 bands.

FIG. 7—Immunoadsorption of anti-AQP1 antibodies from patient sera

AQP1 was immobilized on ELISA plate well (0.1 μg/well); to the wells was added anti-AQP1 positive serum. After incubation, the treated serum was collected and tested by RIPA with indirectly ¹²⁵I-labeled AQP1. In parallel, an equal quantity of the untreated serum was tested by RIPA in order to determine the percentage of the serum anti-AQP1 antibodies which were eliminated from the treated serum.

FIG. 8—Representative sequence of human aquaporin-1

FIG. 9A—Representative nucleotide sequence of human aquaporin-2. This sequence is also shown in GenBank Accession number: Z29491.

FIG. 9B—Representative amino acid sequence of human aquaporin-2 polypeptide. This sequence is also shown in GenBank Accession number: AAD38692.1.

FIG. 10A—Representative nucleotide sequence of human aquaporin-5. This sequence is also shown in GenBank Accession number: U46569.

FIG. 10B—Representative amino acid sequence of human aquaporin-5 polypeptide. This sequence is also shown in GenBank Accession number: AAH32946.1.

FIG. 11A—Representative nucleotide sequence of human aquaporin-7. This sequence is also shown in GenBank Accession number: AB006190.

FIG. 11B—Representative amino acid sequence of human aquaporin-7 polypeptide. This sequence is also shown in GenBank Accession number: CAI13309.1.

FIG. 12A—Representative nucleotide sequence of human aquaporin-8. This sequence is also shown in GenBank Accession number: AB013456.T

FIG. 12B—Representative amino acid sequence of human aquaporin-8 polypeptide. This sequence is also shown in GenBank Accession number: AAF19050.1.

### DETAILED DESCRIPTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, In Press; D. M. J. Lilley and J. E. Dahlberg, 1992, Methods of Enzymology: DNA Structure Part A: Synthesis and Physical Analysis of DNA Methods in Enzymology, Academic Press; and E. M. Shevach and W. Strober, 1992 and periodic supplements, Current Protocols in Immunology, John Wiley & Sons, New York, N.Y. Each of these general texts is herein incorporated by reference.

Aquaporin proteins are made up of six transmembrane α-helices arranged in a right-handed bundle, with the amino and the carboxyl termini located on the cytoplasmic surface of the membrane (Gonen T, Walz T (2006) “The structure of aquaporins”. Q. Rev. Biophys. 39 (4): 361-96; Fu D, Lu M (2007) “The structural basis of water permeation and proton exclusion in aquaporins”. Mol. Membr. Biol. 24 (5-6): 366-74).

The amino and carboxyl halves of the sequence show similarity to each other, in what appears to be a tandem repeat. There are also five interhelical loop regions (A-E) that form the extracellular and cytoplasmic vestibules. Loops B and E are hydrophobic loops that contain the highly, although not completely conserved, asparagine-proline-alanine (NPA) motif, which overlap the middle of the lipid bilayer of the membrane forming a 3-D ‘hourglass’ structure where the water flows through. This overlap forms one of the two well-known channel constriction sites in the peptide, the NPA motif and a second and usually narrower constriction known as ‘selectivity filter’ or ar/R selectivity filter.

Aquaporins form tetramers in the cell membrane, with each monomer acting as a water channel (Gonen T, Walz T (2006) Q. Rev. Biophys. 39 (4)). The different aquaporins contain differences in their peptide sequence, which allows for the size of the pore in the protein to differ between aquaporins. The resultant size of the pore directly affects what molecules are able to pass through the pore, with small pore sizes only allowing small molecules like water to pass through the pore.

Examples of a nucleotide sequence encoding a human aquaporin-4 polypeptide are shown in GenBank Accession Nos. U63622 and U63623. The predicted amino acid sequences of representative human aquaporin-4 polypeptides are shown in GenBank Accession Nos. AAG17964, AAB26957, AAB26958, and 139178. Nucleic acid and amino acid sequences encoding aquaporin-4 from other organisms can be found by searching the GenBank database (www.ncbi.nlm.nih.gov) using “aquaporin-4” as the search word.

Example of a nucleotide sequence encoding a human aquaporin-1 polypeptide is shown in GenBank Accession Nos. AAH22486. The predicted amino acid sequence of representative human aquaporin-1 polypeptide is shown in ProteinBank Accession Nos. AAX24129.1. Nucleic acid and amino acid sequences encoding aquaporin-1 from other organisms can be found by searching the GenBank database (www.ncbi.nlm.nih.gov) using aquaporin-1 as the search word.

Examples of a nucleotide sequence encoding human aquaporins-2, -5-7-8 polypeptide are shown in GenBank Accession No. Z29491, 046569, AB006190 and AB013456 respectively. The representative amino acid sequence of human aquaporins-2, -5-7, -8 polypeptide is shown in GenBank Accession No. AAD38692.1, AAH32946.1, CAI13309.1 and AAF19050.1 respectively. Nucleic acid and amino acid sequences encoding aquaporins-2, -5-7, -8 from other organisms can be found by searching the GenBank database (www.ncbi.nlm.nih.gov) using “aquaporins-2, -5-7, -8” as the search words.

A “protein” or “polypeptide” is understood within the scope of the invention to include single-chain polypeptide molecules, as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. Portions of the polypeptide may be referred to as a “subunit” or a “domain” or “fragment” as applicable. The term polypeptide encompasses polypeptides that have been modified. The term polypeptide also encompasses variants, derivatives, analogues and homologues.

The AQP1 polypeptide referred to herein encompasses variants, derivatives, analogues and homologues of AQP1 for which AQP1 autoantibodies have specific affinity. Similarly, a fragment of an AQP1 polypeptide referred to herein encompasses fragments for which AQP1 autoantibodies have specific affinity.

Similarly, the AQP2, AQP5, AQP7 and AQP8 polypeptides referred to herein encompass variants, derivatives, analogues and homologues of AQP2, AQP5, AQP7 and AQP8 for which respective AQP2, AQP5, AQP7 and AQP8 autoantibodies have specific affinity. Similarly, fragments of AQP2, AQP5, AQP7 and AQP8 polypeptides referred to herein encompass fragments for which respective AQP2, AQP5, AQP7 and AQP8 autoantibodies have specific affinity.

The AQP1 autoantibodies may bind (e.g. via the antigen-specific binding site(s) or paratopes of the antibody, which are present within the variable regions) to an antigenic epitope present within the AQP1 polypeptide. Similarly, the AQP2, AQP5, AQP7 and AQP8 autoantibodies may bind (e.g. via the antigen-specific binding site(s) or paratopes of the antibody, which are present within the variable regions) to an antigenic epitope present within the respective AQP2, AQP5, AQP7 and AQP8 polypeptides. Typically the antibody may bind to the respective AQP1, AQP2, AQP5, AQP7 or AQP8 polypeptide or fragment with high affinity, e.g. with a dissociation constant (K_{d}) of less than 1 μM, preferably less than 10 nM, preferably less than 1 nM. Preferably the AQP1 antibody specifically binds to AQP1 and does not significantly bind unrelated antigens. Preferably the AQP2 antibody specifically binds to AQP2 and does not significantly bind unrelated antigens. Preferably the AQP5 antibody specifically binds to AQP5 and does not significantly bind unrelated antigens. Preferably the AQP7 antibody specifically binds to AQP7 and does not significantly bind unrelated antigens. Preferably the AQP8 antibody specifically binds to AQP8 and does not significantly bind unrelated antigens.

AQP1, AQP2, AQP5, AQP7 and AQP8 polypeptides or fragments thereof include one or more epitopic sites. Computer algorithms are available for predicting binding epitopes, e.g., MHC-I and MHC-II binding epitopes. See, for example, http://bimas.dcrt.nih.gov:80/molbio/hla_bind/(Parker et al., J. Immunol., 152:163 (1994); Southwood et al., J. Immunol., 160:3363 (1998)).

B-cell epitopes of a protein are mainly localised at its surface. To predict B-cell epitopes, two well-known methods may be used: 2D-structure prediction and antigenic index prediction. The 2D-structure prediction can be made using the psipred program (from David Jones, Brunel Bio-informatics Group, Dept. Biological Sciences, Brunel University, Uxbridge UB8 3PH, UK). The antigenic index can be calculated on the basis of the method described by Jameson and Wolf (CABIOS 4: 181-186 [1988]). In addition, as explained on page 73, lines 2 to 5, the prediction of useful T-helper cell epitopes can be performed using the T-epitope method described by Sturniolo et al (Nature Biotech. 17: 555-561 [1999]). Hirschberg et al (APMIS Volume 99, Issue 1-6, pages 515-520, January 1991) demonstrates the use of these techniques to generate peptide fragments of the Mycoplasma pneumoniae P1 protein which were recognised by antibodies in the serum of patients.

The epitopic site allows immunologic detection of AQP1, AQP2, AQP5, AQP7 and AQP8 autoantibodies in sera with reasonable assurance. Usually, it is desirable that the epitopic site be antigenically distinct from other closely related antigens (e.g., other members of a family of polypeptides). A representative antigenic fragment can include, for example, the extracellular domain of a membrane-bound protein.

A variant or derivative sequence can be obtained by addition, deletion, substitution, modification, replacement and/or variation of at least one residue present in the naturally-occurring protein.

The term “analogue” as used herein, in relation to polypeptides includes any mimetic, that is, a chemical compound that possesses at least one of the endogenous functions of the polypeptides which it mimics.

Typically, amino acid substitutions may be made, for example from 1, 2 or 3 to 10 or 20 substitutions provided that the modified sequence retains the required activity or ability. Amino acid substitutions may include the use of non-naturally occurring analogues.

Polypeptides for use in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the transport or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

The AQP1, AQP2, AQP5, AQP7 and/or AQP8 polypeptides and fragments may be labeled directly or indirectly with a second substance that provides for detectable signal. Examples of labels include, but are not limited to radioisotopes, enzymes, substrates, cofactors, inhibitors, fluorescers, chemiluminescers, magnetic particles, and the like.

The presence of AQP1, AQP2, AQP5, AQP7 and/or AQP8 autoantibodies can be used to diagnose diseases associated with the presence or elevated expression of said antibody or said antibodies, such as diseases characterised by demyelinated lesions, in particular demyelinated lesions of the CNS or of the peripheral nervous system, at an early stage of the disease before all clinical criteria are fulfilled, thus justifying early initiation of appropriate immunosuppressant therapy. The presence of AQP1, AQP2, AQP5, AQP7 and/or AQP8 autoantibodies may also be used to distinguish the autoimmune disease from other autoimmune diseases such as, but not limited to classic MS and MG.

Detecting AQP1, AQP2, AQP5, AQP7 and/or AQP8 autoantibodies also can provide a quantifiable biomarker for monitoring disease progression and response to therapy. In addition, the identification of an antibody associated with AQP1, AQP2, AQP5, AQP7 or AQP8 provides a valuable tool for developing animal models (e.g., by passive transfer of NMO-specific antibodies or active immunization with AQP1, AQP2, AQP5, AQP7 and/or AQP8 antigenic polypeptides or DNA vaccines encoding such antigenic polypeptides) to investigate the pathogenesis of diseases characterised by AQP1, AQP2, AQP5, AQP7 and/or AQP8 autoantibody expression and to test potential new therapies.

The present invention provides for methods of detecting AQP1, AQP2, AQP5, AQP7 and/or AQP8 specific autoantibodies in an individual. Individuals for whom the methods of the invention might be used typically may present abnormal neurological symptoms including, but not limited to, tingling, numbness, weakness, limb spasms, limb paralysis, sensory loss, radicular pain, diplopia and nystagmus, vision impairment, loss of bladder and/or bowel control, or other neurological symptoms of unknown origin.

Further provided by the invention are kits containing an AQP1, AQP2, AQP5, AQP7 and/or AQP8 polypeptide or fragment thereof. The kit may further include a second substance that provides for detectable signal. A kit typically also includes directions for using the AQP1, AQP2, AQP5, AQP7 and/or AQP8 polypeptide of fragment for detecting the respective AQP1, AQP2, AQP5, AQP7 and/or AQP8-specific autoantibodies in a biological sample.

The method describes an association between the presence of abnormal levels or pattern of expression of the AQP1, AQP2, AQP5, AQP7 and/or AQP8, the subsequently produced AQP1, AQP2, AQP5, AQP7 or AQP8-specific autoantibody or autoantibodies, and the resulting disorder in a subject.

Antibodies may be produced by standard techniques. Polyclonal, monoclonal, chimeric, single chain, Fab fragments, fragments produced by a Fab expression library, as well as mimetics thereof may be used in the present invention. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies.

If polyclonal antibodies are desired, a selected mammal (e.g., mouse, rabbit, goat, horse, etc.) is immunised with the relevant antigen. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. The polyclonal antibodies can be purified by immunoaffinity chromatography. Techniques for producing and processing polyclonal antisera are known in the art.

Monoclonal antibodies can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies may be prepared using any technique which provides for the production of antibody molecules by continuous cell lines in culture. These include, but are not limited to, the hybridoma technique originally described by Koehler and Milstein (Koehler and Milstein, 1975), the human B-cell hybridoma technique (Kosbor et al., 1983; Cote et al., 1983) and the EBV-hybridoma technique (Cole et al., 1985). In addition, techniques developed for the production of “chimeric antibodies”, the splicing of mouse antibody genes to human antibody genes to obtain a molecule with appropriate antigen specificity and biological activity can be used (Morrison et al., 1984; Neuberger et al., 1984; Takeda et al., 1985). Alternatively, techniques described for the production of single chain antibodies (U.S. Pat. No. 4,946,779) can be adapted to produce the substance specific single chain antibodies.

Within certain embodiments, the use of antigen-binding fragments of antibodies may be preferred. Such fragments include Fab fragments, which may be prepared using standard techniques, for example digestion by papain to yield Fab and Fc fragments which may be separated by affinity chromatography.

A “sample” is understood within the scope of the invention to refer to a sample which is derived from a subject. The biological sample may be obtained directly from the subject or may be derived from cultured cells obtained from said subject. Preferred samples are those derived from blood which may comprise plasma, serum or fractions such as an immunoglobulin fraction. The biological sample may have been subjected to a treatment such as dilution in a carrier.

Assays used in the diagnosis or prognosis methods described herein include, but are not limited to, an immunoassay, a ligand-binding assay, a surface plasmon resonance assay or a microchip-based assay or any other assay commonly known in the art. Details regarding such assays can be found in known literature and periodic supplements, such as Current Protocols in Immunology, Copyright© 2010 by John Wiley and Sons, Inc, Last Updated: Feb. 8, 2010, Print ISSN: 1934-3671 (see e.g. chapters 2, 6 and 10), which is hereby incorporated by reference.

An “immunoassay” is understood within the scope of the invention to include an enzyme-linked immunosorbent assay (ELISA), radioimmunoassay (RIA), immunoprecipitation assay, immunofluorescence assay (IFA), indirect immunofluorescence assay (IIF), enzyme linked assay (EIA), luminescence immunoassay (LIA), Western Blot assay (WB) and cell based assay (CBA) or any other immunoassay commonly known in the art.

Further provided by the invention are methods of treating a subject having a disease characterised by the presence of, or elevated expression of, AQP1, AQP2, AQP5, AQP7 and/or AQP8 antibodies. Methods of treating a subject include, but are not limited to, apheresis and T cell receptor-based immunotherapy.

Methods and extracorporeal systems for apheresis (i.e., the process of withdrawing blood from an individual, removing components from the blood, and returning the blood, or blood depleted of one or more components, to the individual) are known in the art (see, for example, U.S. Pat. Nos. 4,708,713; 5,258,503; 5,386,734; and 6,409,696). The present invention provides a method of removing AQP1, AQP2, AQP5, AQP7 and/or AQP8-specific autoantibodies from a body fluid of an individual. The method involves withdrawing a body fluid from a subject; removing a substantial portion of AQP1, AQP2, AQP5, AQP7 and/or AQP8-specific autoantibodies from the fluid; and returning the fluid to the subject. Antibodies removed can be of any class, e.g., IgG (such as IgG1, IgG2, IgG3, IgG4), IgM, IgD, IgA, or IgE antibodies.

As used herein, a “substantial portion” means removing at least 20%, 30%, 40%, 50%, 60%, 70%, 80%, 90%, 95%, 96%, 97%, 98%, 99%, or even 100% of the AQP1, AQP2, AQP5, AQP7 or AQP8-specific autoantibodies that were present in the body fluid prior to removal. The body fluid can be blood plasma or any other body fluid, e.g., lymph or cerebrospinal fluid.

Removal of AQP1, AQP2, AQP5, AQP7 and/or AQP8-specific autoantibodies may be performed by contacting a body fluid with an AQP1, AQP2, AQP5, AQP7 and/or AQP8 polypeptide or fragment thereof. The AQP1, AQP2, AQP5, AQP7 and/or AQP8 polypeptide or fragment can be bound to a solid support. Such solid supports can be, without limitation, membranes, fibres, spherical beads, or granules and can be made with a water-insoluble, preferably porous, biocompatible material, e.g., organic polymers such as agarose, dextran, and polyacrylamide, or inorganic porous materials such as porous glass or porous silica gel. Such materials are suitable or can be adapted (e.g., derivatized with appropriate chemical groups) for attachment of AQP1, AQP2, AQP5, AQP7 and/or AQP8 polypeptide or fragment thereof.

An effective amount of an AQP1, AQP2, AQP5, AQP7 and/or AQP8 polypeptide or fragment may be administered to a subject to modulate the subject's AQP1-mediated immune response. Thus the AQP1 polypeptide or fragment may be used in immunotherapy to treat a disorder characterised by the presence of AQP1 autoantibodies.

Thus, there is provided a pharmaceutical composition comprising an AQP1, AQP2, AQP5, AQP7 and/or AQP8 polypeptide or fragment.

A pharmaceutical composition of the invention may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s), and other carrier agents that may aid or increase the viral entry into the target site (such as for example a lipid delivery system).

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Methods to deliver a composition to the brain are known in the art. For example, a composition of the invention can be modified by attaching a ligand (e.g., an antibody or antibody fragment) that recognizes a brain-specific or neuron-specific receptor. In addition, methods of enhancing transport of molecules across the blood-brain barrier are known, and take advantage of passive diffusion (e.g., using electromagnetic fields, nitric oxide donors or sodium caprate) or receptor-mediated endocytosis (e.g., attachment of the virus particle to, for example, an anti-transferrin antibody or to putrescine). Brain-specific or neuron-specific promoter and/or transcriptional regulatory elements may also be used (see, for example, U.S. Pat. No. 5,976,872 or 6,066,726).

A “subject” refers to either a human or non-human animal. Examples of non-human animals include vertebrates, e.g., mammals, such as non-human primates (particularly higher primates), dogs, rodents (e.g., mice, rats, or guinea pigs), pigs and cats, etc. In a preferred embodiment, the subject is a human.

Further preferred features and embodiments of the present invention will now be described by way of the following non-limiting examples.

### Example 1

### Methods

### Patient Sera

Sera of 362 patients for whom test for anti-AQP4 antibodies was requested by their doctors, were used after informed consent; The selection of these sera was as follows: 297 sera were from almost all patients who requested in our diagnosis unit testing for NMO antibodies, independently of their anti-AQP4 outcome (35 of these sera were anti-AQP4 positive and 5 ambiguous). The remaining 65 sera were selected as either anti-AQP4 positive (49 sera) or anti-AQP4 ambiguous (16 sera). 110 of the 362 sera were from patients evaluated clinically at the Aiginiteion University Hospital. In addition to the above 362 sera, 20 patients with classical MS, 51 MG patients (positive for anti-acetylcholine receptor antibodies), and 100 sera from healthy controls were also tested for the presence of anti-AQP1 antibodies. Retrospective analysis, of MRI, clinical, cytologic and statistical data, from patients positive for anti-AQP1 antibodies with available data followed.

### AQP1, AQP2, AQP5, AQP4, AQP7 and AQP8

Human recombinant AQP4 with 6-his tag was expressed in Pichia pastoris expression system and purified by Ni-column chromatography and gel filtration. Human recombinant AQP1 with glutaphion-S-transferase (GST) tag was purchased from Novus Biologicals; it was expressed in wheat germ cell-free protein expression system and purified by affinity purification with immobilized glutathione. Human recombinant AQP2, AQP7, and AQP8 proteins with a glutathione-S-transferase (GST) tag expressed in the wheat germ cell-free protein expression system and purified by affinity chromatography were purchased from Novus Biologicals, Cambridge, UK. AQP5 cDNA clone was purchased from OriGene Technologies Inc, Rockville, Md., and was transiently expressed in HEK293 cells.

### Radioimmunoprecipitation Assay (RIPA)

RIPA usually involves the incubation of the test serum with radiolabeled antigen, followed by the addition of an anti-Ig serum to make an Ig/anti-Ig precipitate; if the test serum has antibodies against the labeled antigen, the latter will be coprecipitated with the Ig complex resulting in radioactive precipitate. In our RIPA, recombinant human AQP1 or AQP4 was ¹²⁵I-labeled indirectly as follows: 0.25 nmole AQP were incubated with 5 nmole biotin. The biotinilated AQP was indirectly labeled by incubation with ¹²⁵I-streptavidin at a ratio: 0.1 μg biotinilated-AQP with 50,000 cpm ¹²⁵I-streptavidin, for 1 hr on ice. For the RIPA, 5 μl (or less) of test serum were incubated for 2 h at 25° C. with 50 μl phosphate buffered saline (PBS)-0.5% Triton X-100 buffer, with 0.1 μg of the indirectly labeled AQP. After 2 h incubation at room temperature, 50 μl of goat anti-human immunoglobulin antiserum was added and after incubation for 2 h at 25° C. the formed pellet was washed twice with 1 ml of assay buffer (0.5% Triton X-100 in PBS). The radioactivity in the washed pellet was counted in a γ-counter.

The average of the cpm values for the healthy human control sera (256 cpm), plus 3SD (3×35 cpm), total 361 cpm, multiplied by 1.4 (total 361×1.4=505 cpm) was considered the cut-off for positive sera whereas multiplied by 1.1 (361×1.1=397 cpm) was considered the cut-off for “ambiguous” sera. Each 100 cpm above the background corresponds to 1 nM antibody when using 5 μl serum in the assay, therefore the ambiguous and positive cut-offs correspond to 1.4 nM (397 cpm=256 cpm average background+141 extra cpm; 141/100=≈1.4 nM) and 2.5 nM (505 cpm=256 cpm average background+249 extra cpm; 249/100=≈2.5 nM) antibody concentrations.

Ten sera positive in this assay were also tested for binding to the same amount of 125I-streptavidin in the absence of biotinilated aquaporin. They precipitated background cpm values, similar to those of the NHS controls, confirming that the antibodies indeed bind to the AQP1-GST rather than to the labeled streptavidin. To exclude the possibility that some antibodies bound to the GST moiety of the AQP1-GST fusion protein, 9 anti-AQP1 and 4 anti-AQP4 sera were preincubated for 3 hr with excess GST immobilized on Sepharose-glutathione beads (10 μl serum with 1.6 μg immobilized GST); then 5 μl of the treated sera were tested in RIPA with labeled AQP1 or AQP4. No decrease in the bound cpm was observed confirming that the patients' antibodies indeed bind to the AQP1 part of the fusion protein.

Finally, in order to verify the reason that AQP1-positive sera pretreated with liver powder cannot give positive result in IIF, two anti-AQP1 and two anti-AQP4 serum samples (5 μl each diluted in 100 μl buffer) were pretreated with guinea pig liver powder (20 mg each) for 1 h. After centrifugation the supernatants were tested with labeled AQP1 and AQP4 respectively and the precipitated cpms were compared with those of the untreated sera (similarly diluted).

### Two-Step RIPA

In selective cases, a more sensitive radioimmunoassay, based on a general very sensitive immunoassay method we developed, as described in PCT/IB2011/001014, which is herein incorporated by reference, involving two steps (two-step RIPA) was also used:

Step 1: ELISA plates were plated with 50 pi carbonate buffer pH 9.6 containing 0.1 μg AQP1 or AQP4 per well, incubated for 2 hr at 25° C., followed by saturation with 1% PBS-bovine serum albumin (PBS-BSA) for 1 h at 25° C. Then, 0.15 ml of test serum was added to each well and the sample incubated overnight at 4° C. with constant rotation. The wells were sequentially washed 3 times with PBS and once with 0.1 M glycine-HCl, pH 6. Then 50 μl of 0.1 M glycine-HCl buffer, pH 2.5, was added to the washed wells and shaked for 1 min. The liquid, containing the released autoantibodies was then transferred to test tubes containing 10 μl of 0.375 M Tris buffer, pH 8.8, in order to immediately neutralize the pH.

Step 2: To the above mixture, 20 μl of buffer containing 0.1 μg biotinilated AQP and 50.000 cpm ¹²⁵I-labeled streptavidin) and 2 μl of carrier NHS were added. After 6 h incubation at room temperature, 50 μl of goat anti-human immunoglobulin antiserum was added. After incubation for 2 h, the formed pellet was washed twice with 1 ml of PBS-0.5% Triton X-100 and the radioactivity of the washed pellet counted in a γ-counter.

### Characterization of the Autoantigen by Immunoprecipitation and Western Blot Assay

Anti-AQP1 and anti-AQP4 positive sera and NHS were incubated with 0.1 μg preparation of unlabeled biotinilated AQP1 or AQP4 respectively. Then, the mixtures were incubated with 10 μl Sepharose beads with immobilized protein A. The beads were subsequently washed, treated with sample buffer for PAGE containing 2% SDS, 50 mM Tris-HCl pH 6.8, 10% glycerol, 0.1% bromophenol blue and 2% β-mercaptethanol to release the bound antibodies with any bound biotinilated AQP and subjected to 4-12% gradient polyacrylamide gel electrophoresis (PAGE) and subsequently blotted to nitrocellulose membrane. The detection was accomplished by incubating the membranes with 1/500 dilution of HRP-streptavidin for 16 h at 4° C. followed by saturation with 3% PBS-BSA for 1 h at 25° C.

In parallel, 0.01 and 1 μg of biotinilated AQP1 were directly run on PAGE, blotted and detected as above with HRP-streptavidin in order to semi-quantitate the protein.

### Indirect Immunofluorescence

Immunofluorescence microscopy on frozen nonpathological mouse whole brain sagital sections on slides is performed. Specifically, sections are incubated with 10% formalin in PBS for 4 min for fixation, followed by three washes in PBS. Next, blocking is performed, using 10% normal horse serum in PBS for 1 hr. Then, sections are incubated with patient and control sera (1:60) for 1 hr at room temperature. After washing the section three times with PBS, the secondary antibody, Alexa-488 goat-anti-human IgG (1/200 dilution) is applied for 1 hr at room temperature, washed with PBS three times, and viewed on a confocal microscope. Seropositivity is reported when IgG bound selectively to AQP1-rich tissues. Contrary to previously standardized method for detecting anti-NMO IgG (Lennon et al. (2004) Lancet 364(9451): 2106-2112), serum is not preabsorbed with liver powder in order to avoid elimination of the anti-AQP1 antibodies. To overcome the background problem, anti-AQP1 antibodies are preferably, but not necessarily, partially purified by affinity chromatography on ELISA plates with plated AQP1.

### Retrospective Analysis of Available MRI and Clinical Data

For 20 anti-AQP1 positive sera (5 anti-AQP4 positive and 15 anti-AQP4 negative) and 9 anti-AQP1 ambiguous (the 8 of which were anti-AQP4-negative) detailed clinical data of the corresponding patients could be obtained. These included: MRI (brain, spinal cord and optic nerves), clinical (CNS, periphery, etc) and cytological (plasma albumin and CSF) study.

### Example 2

### Antibodies Specific for AQP1 are Present in Patients Suspected for NMO or NMO-Like Symptoms

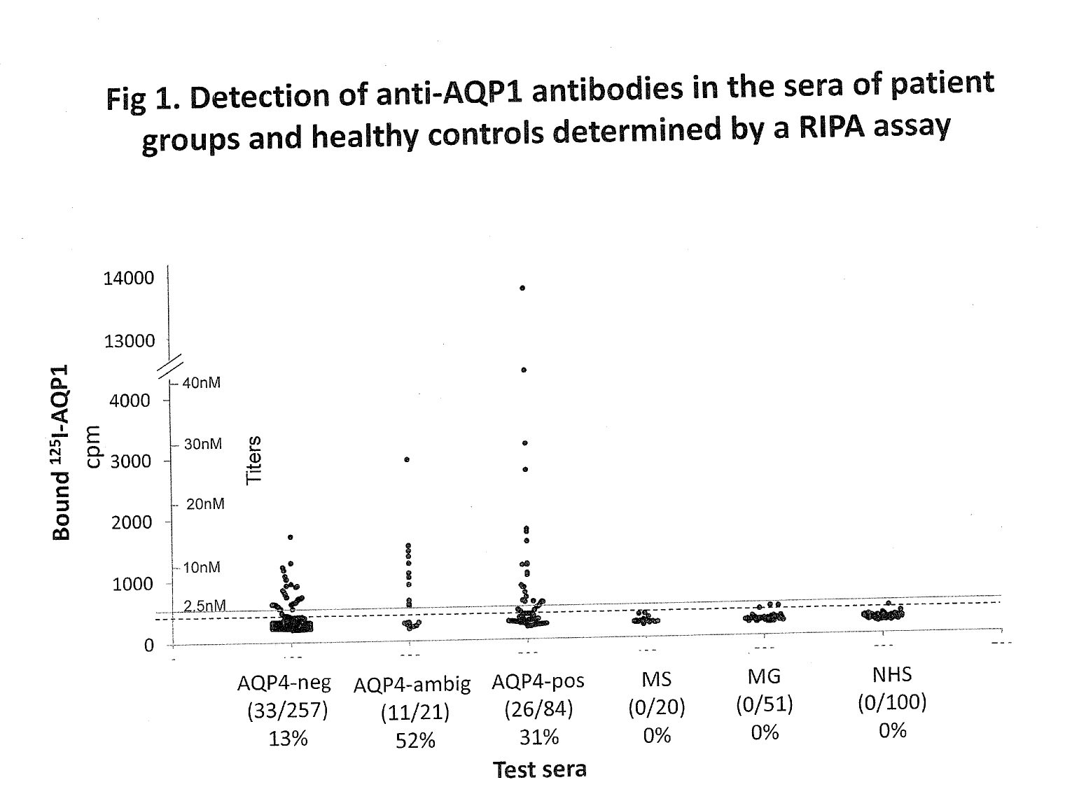

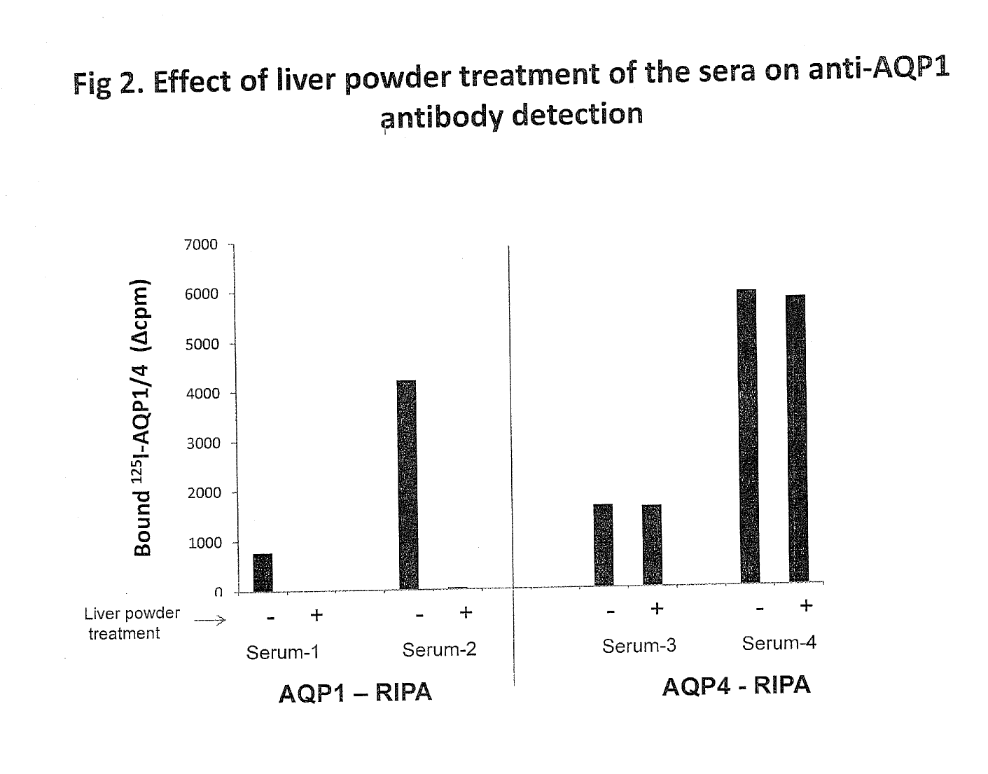

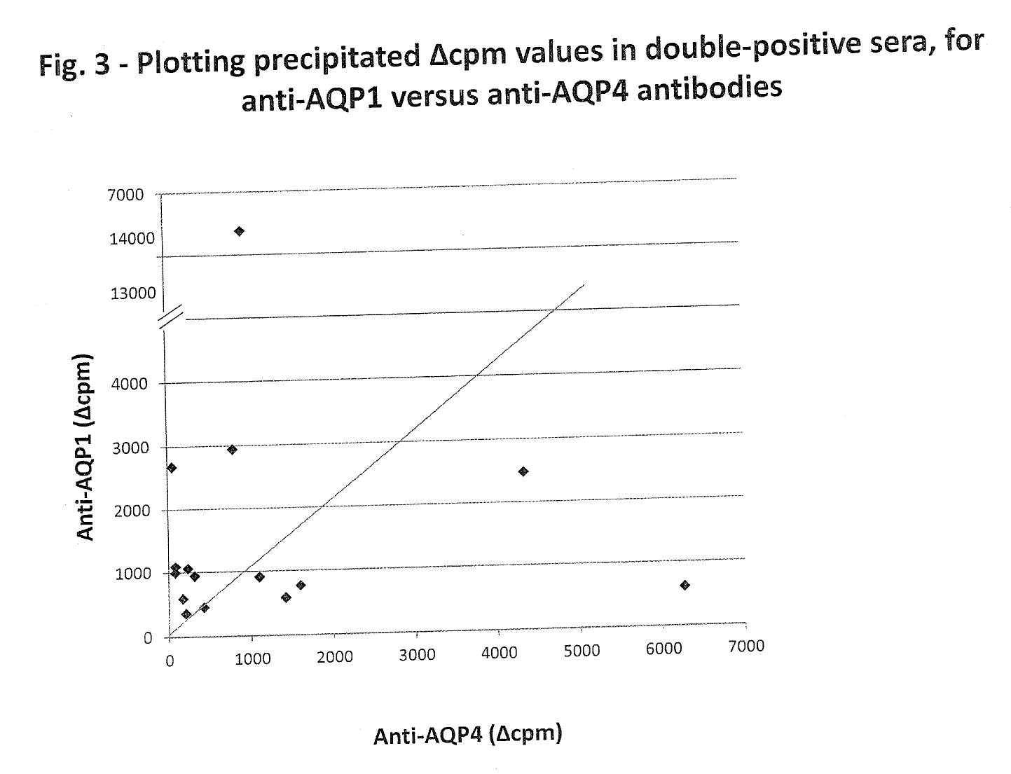

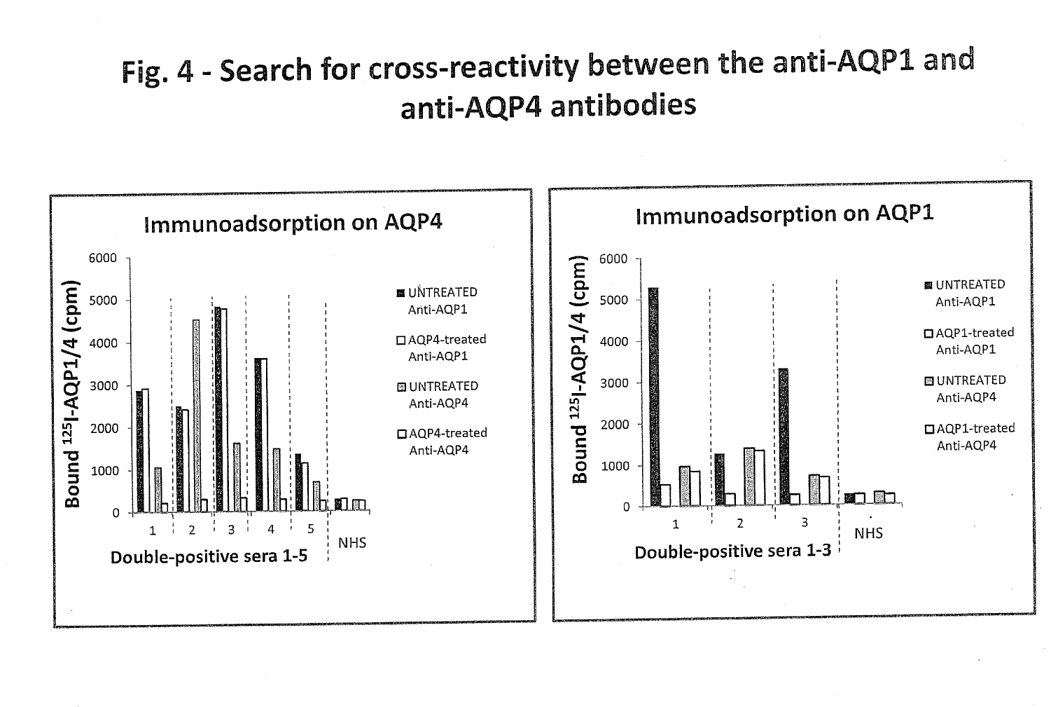

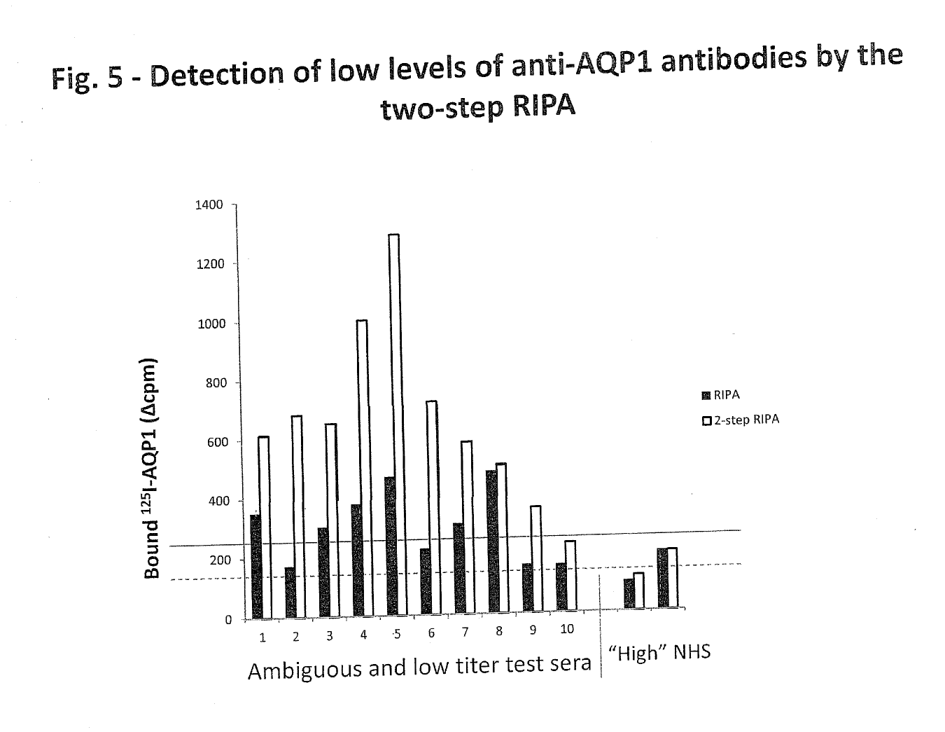

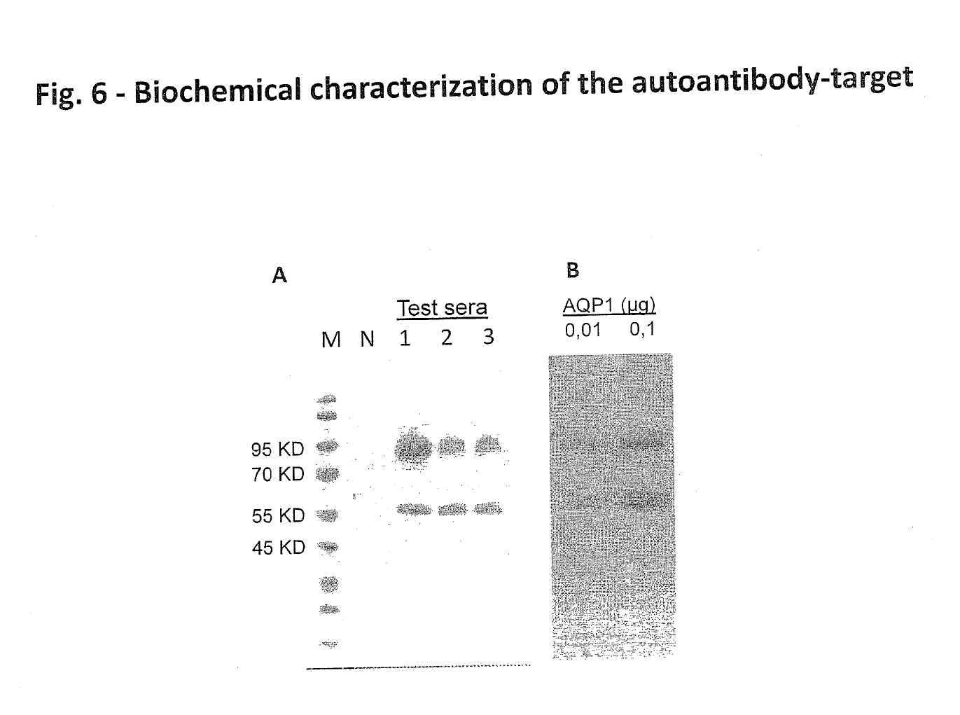

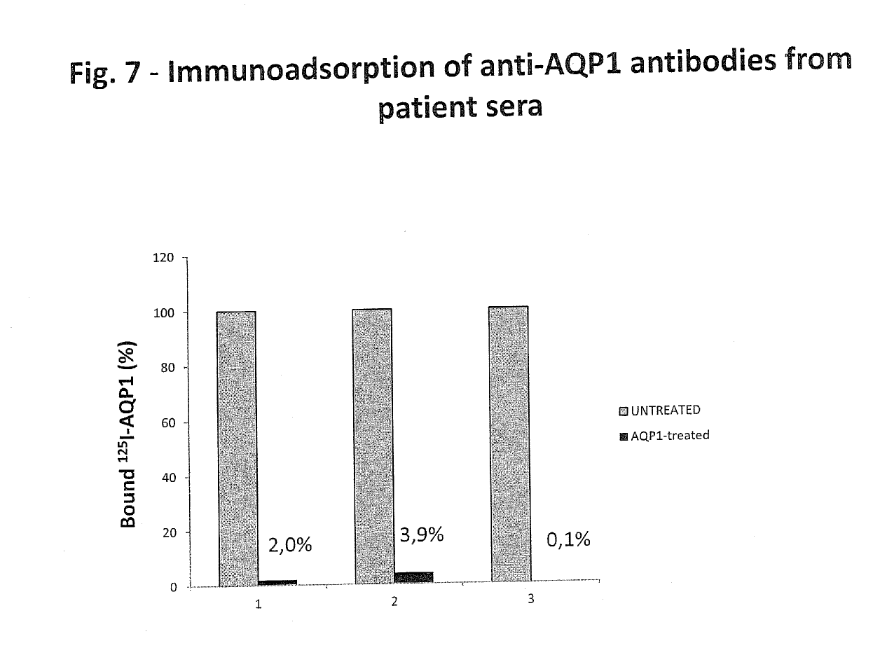

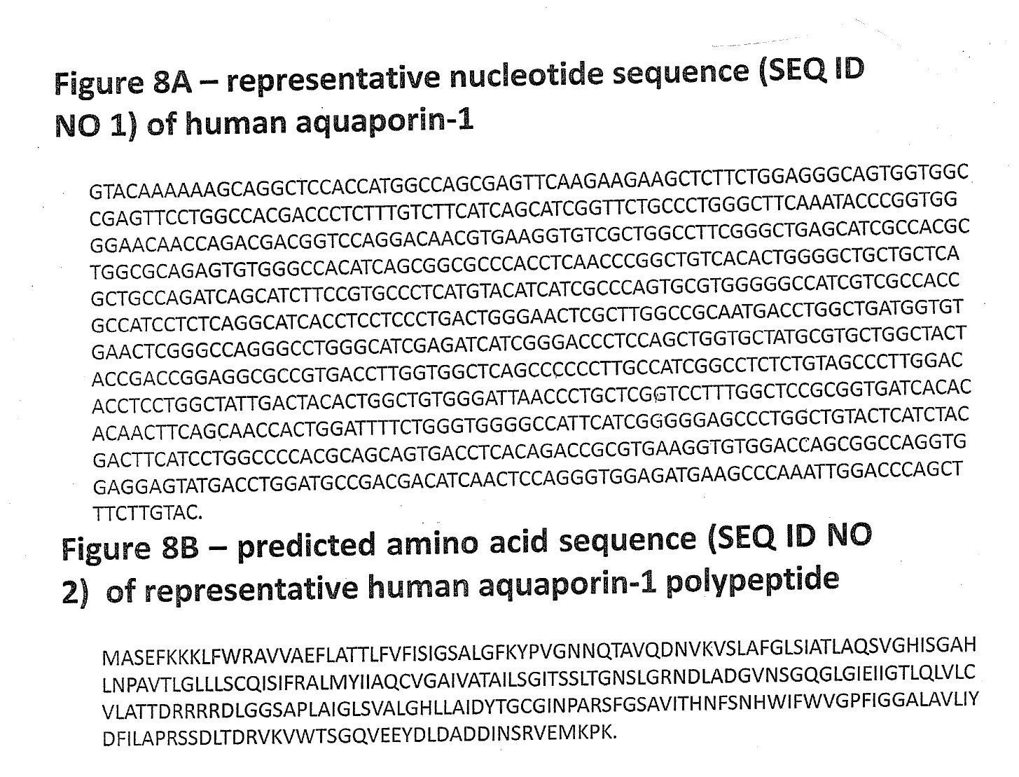

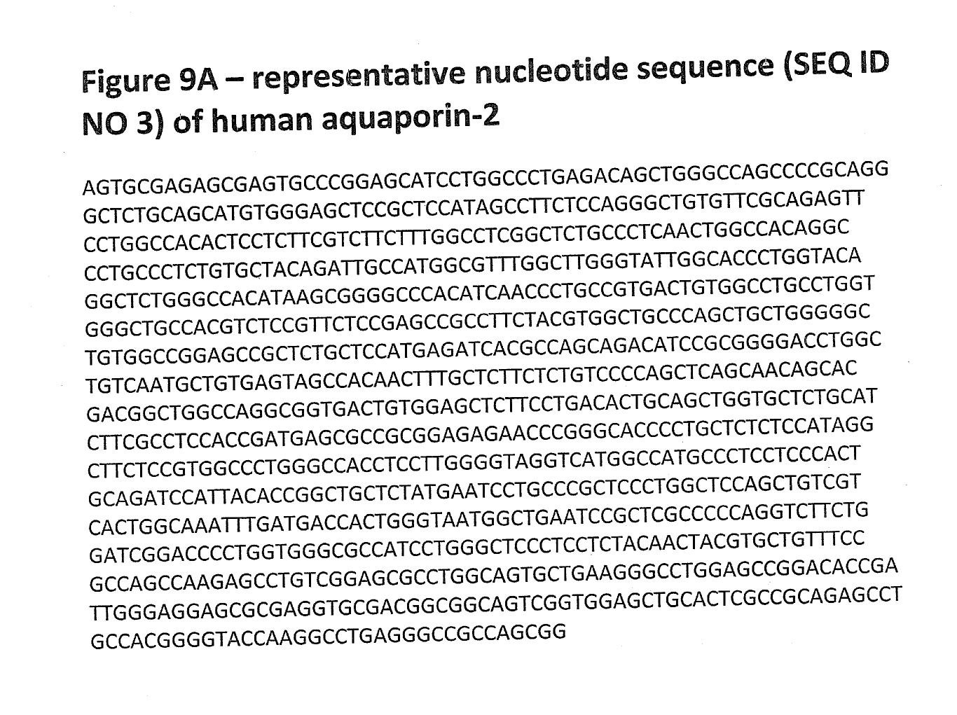

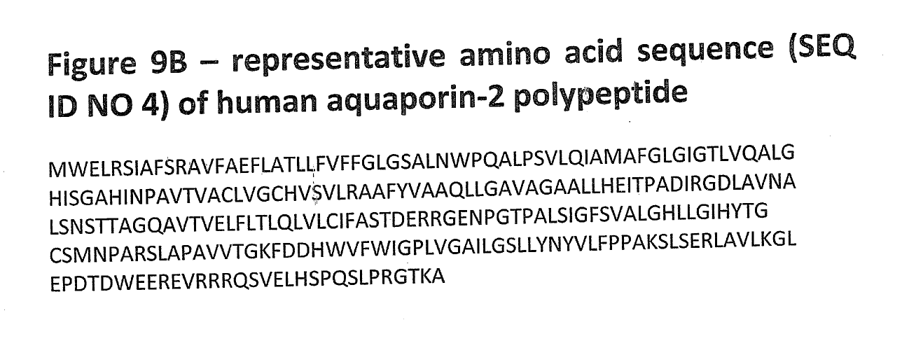

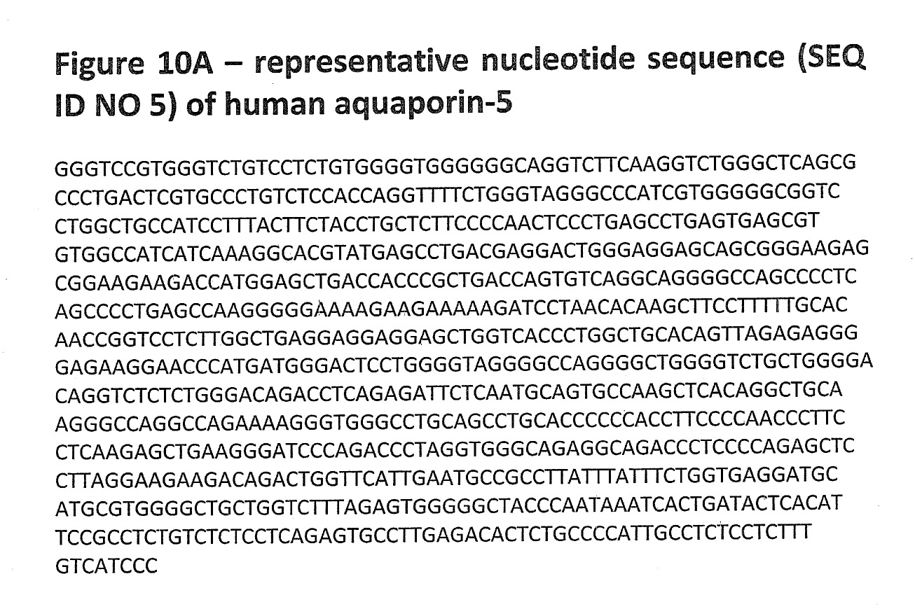

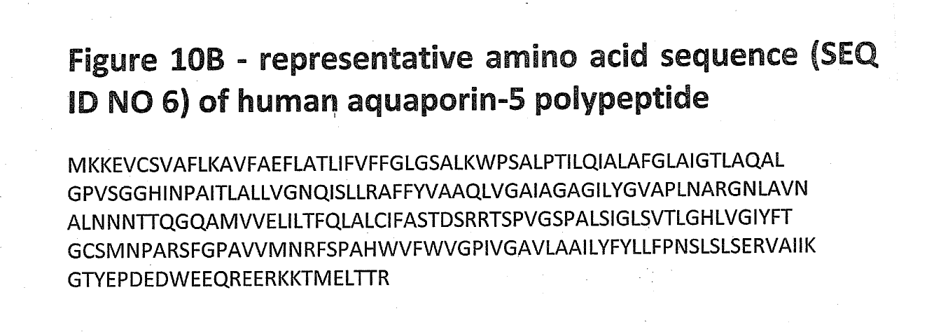

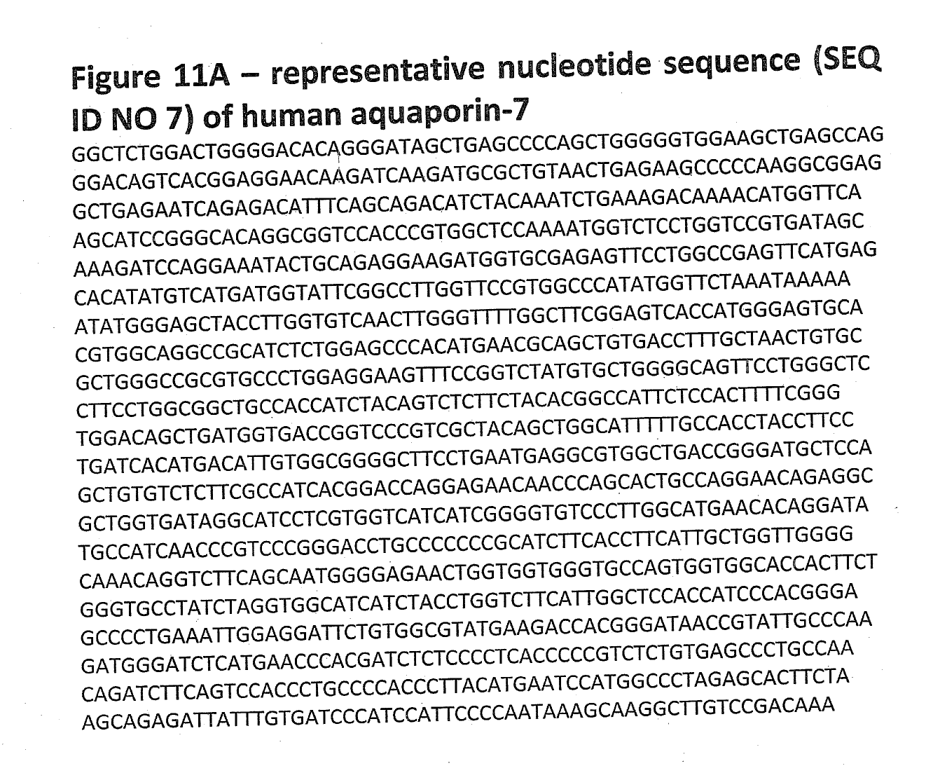

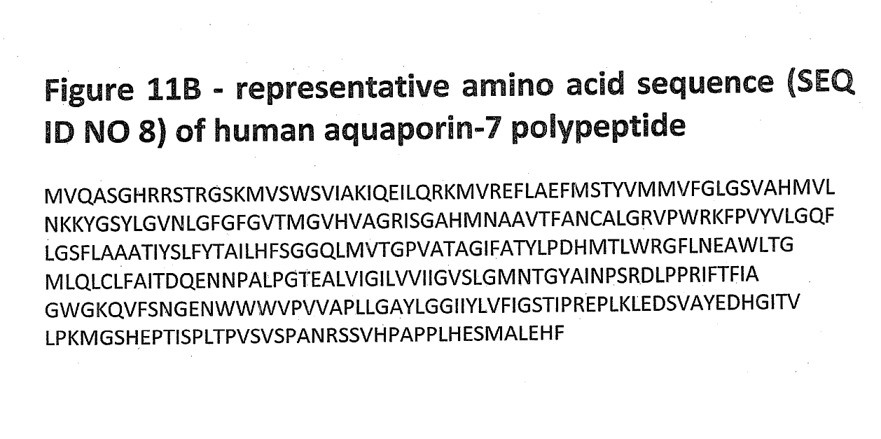

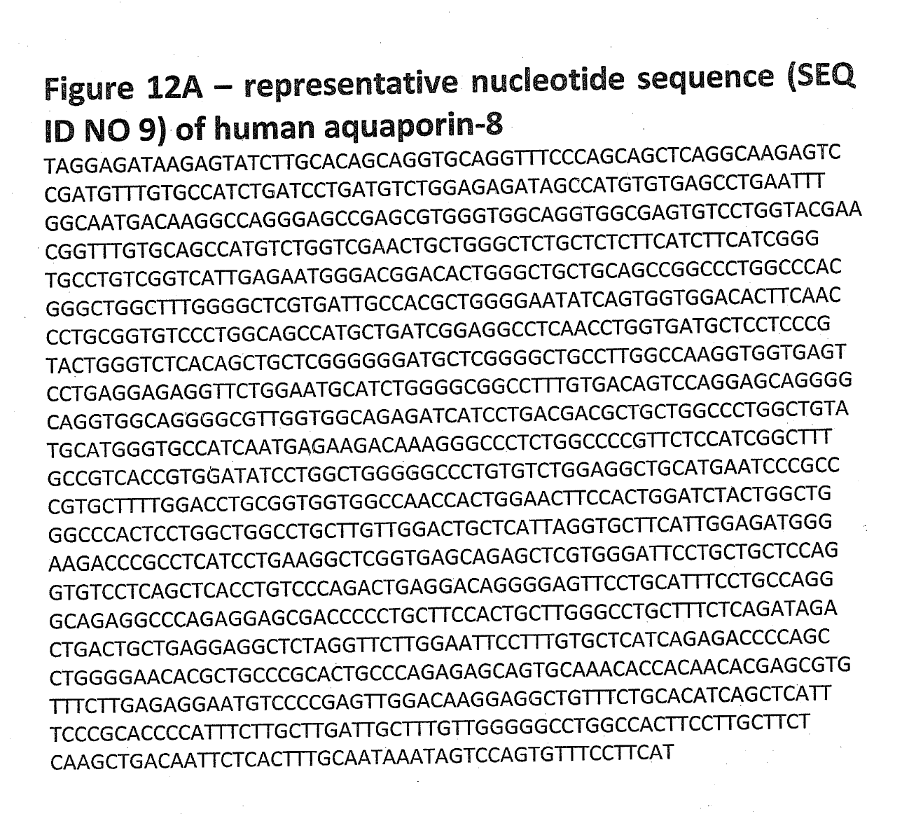

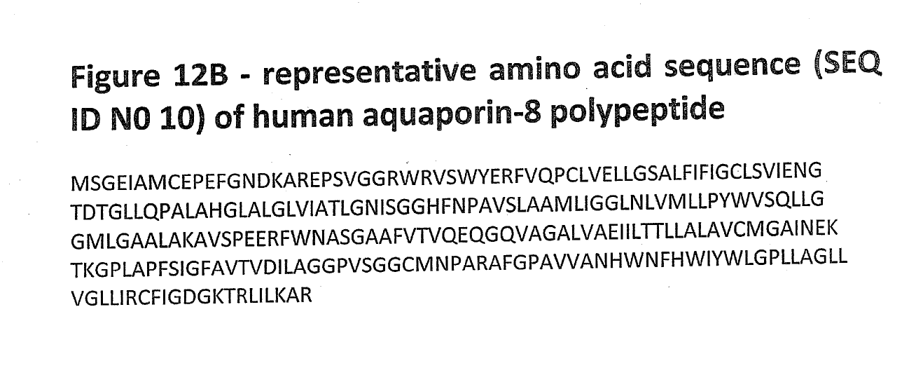

We examined sera from 362 patients, suspected for NMO, with RIPA for anti-AQP1 antibodies. Previously these sera were examined for anti-AQP4 antibodies with the more sensitive two-step RIPA and had identified 84 of them as anti-AQP4 positive. We found in total 70 sera positive for AQP1 antibodies (FIG. 1). We found 44 AQP1-positive sera out of the 278 AQP4-negative or ambiguous (AQP1+/AQP4−; 16%) and 26 AQP1-positive sera out of the 84 AQP4-positive (AQP1+/AQP4+; 31%). The M/F ratios were 1:2.4 (13/31) for the AQP1+/AQP4− patients, 1:4.2 (5/21) for the AQP1+/AQP4+ patients, and 1:4.3 (11/47) for the AQP1−/AQP4+ patients. I.e. males are considerably more frequent in the exclusively AQP1+ group of patients than in the AQP4+ group (with or without anti-AQP1 antibodies), although females remain more frequent in both disease groups.

FIG. 1 also shows that 20 sera from patients with classical MS, 51 sera of MG patients, positive for anti-AChR antibodies, and 100 sera from healthy controls were all negative for anti-AQP1 antibodies (with 0, 3 and 1 sera characterized as ambiguous, respectively). These data suggest that anti-AQP1 antibodies are a specific biomarker for a population of patients with demyelinated lesions.

Two anti-AQP1 positive and two anti-AQP4 positive sera were pretreated with guinea pig liver powder; their supernatants were tested with labeled AQP1 and AQP4, respectively, and compared with those of the untreated sera. It was found that the liver powder practically completely (by 99-100%) adsorbed the anti-AQP1 antibodies whereas it did not adsorb at all the anti-AQP4 antibodies (FIG. 2). This finding is based on the known fact that liver powder contains AQP1, but not AQP4, and shows clearly why AQP1-positive sera pretreated with liver powder cannot easily give positive result in HF. Thus, autoantibodies to AQP1 usually might not be detected with the usual IIF assay on mouse tissues used for NMO-IgG antibodies because this assay, in order to reduce background, usually involves treatment of the test serum with guinea pig liver powder, which incidentally contains AQP1 (Talbot et al. (2003) Cells Tissues Organs 174(3): 117-128) and therefore any autoantibodies to AQP1 in the test serum would be eliminated and not detected.

### Example 3

### There is No Correlation Between Anti-AQP1 and Anti-AQP4 Concentrations in Double Positive (Anti-AQP1+/Anti-AQP4+) Sera

We then tested whether, in the double positive sera, there is any correlation of antibody concentrations between the two groups of autoantibodies, which would be an indication of linked immune response. FIG. 3 plots the anti-AQP1 versus the anti-AQP4 antibody levels as tested by regular RIPAs. It is clear that there is no correlation between the levels of the two antibody groups in the double positive sera. Furthermore, in most cases (9 versus 5) the anti-AQP1 levels were higher than the anti-AQP4 antibody levels. This suggests that the two immune responses are independent rather than the one being byproduct/consequence of the other.

### Example 4

### Search for Cross-Reactivity Between the Two Antibody Groups (Anti-AQP1 and Anti-AQP4)

AQP1 and AQP4 belong to the same protein family and they have extensive amino acid sequence homology. The presence of sera with only anti-AQP1 or only anti-AQP4 antibodies shows that the two groups of antibodies (to AQP1 or AQP4) differ considerably and the binding to the two antigens is not due solely to cross-reactive antibodies. To test for any cross-reactive antibodies, we depleted selected sera from their antibodies binding to the one antigen and tested the depleted sera for binding to either antigen. Specifically, the test sera were treated with immobilized AQP1 or AQP4 (to immunoadsorb all antibodies binding to AQP1 or AQP4 respectively) and subsequently the treated sera were tested by RIPA using ¹²⁵I-labeled AQP1 and AQP4. FIG. 4 shows that the immobilized AQP1 practically completely eliminated the anti-AQP1 antibodies but did not reduce the anti-AQP4 values, and the reverse. Therefore no significant antibody cross-reactivity could be detected in the tested sera.

### Example 5

### Verification of the Anti-AQP1 Antibodies in Low Titer Sera by the Two-Step Assay

In order to easily screen the several hundreds of sera for anti-AQP1 antibodies, in the experiments described above, we used the regular RIPA instead of the more sensitive two-step RIPA we developed recently as described in PCT/IB2011/001014, which is herein incorporated by reference. The two-step RIPA was earlier used for the detection of anti-AQP4 antibodies in these sera. It is likely therefore that some sera with low levels of anti-AQP1 antibodies escaped detection. We here used the potency of the two-step RIPA for the verification of the positivity of some low positive sera, as detected with the regular RIPA. FIG. 5 confirms the positivity of some low positive (no. 1, 3, 4, 5, 7, 8) or ambiguous sera (no. 2, 6, 9), whereas the Acpm value of two “high cpm” NHS (the second had the highest precipitated cpm among the 100 tested NHS) did not increase suggesting that their above the average values are probably due to non-specific factors (e.g. possibly binding to a minor impurity in the AQP1 preparation) rather than to the presence of anti-AQP1 autoantibodies. Also one ambiguous serum (no 10) remained ambiguous by the two-step RIPA suggesting that it may not be really positive.

### Example 6

### Biochemical Characterization of the Autoantibody-Target of the Anti-AQP1 Sera

To further biochemically characterize the target of the anti-AQP1 sera, using three anti-AQP1 positive sera we immunoprecipitated the antibody-antigen complexes and identified the bound antigen by PAGE followed by Western blotting. Specifically, the sera were incubated with biotinilated AQP1 followed by precipitation with Sepharose-protein A beads. The AQP1 indirectly bound on the beads (through the bound autoantibodies) was released, run on PAGE and revealed by Western blot using HRP-streptavidin and its substrate. Semi-quantitation of the precipitated AQP1 was achieved by comparison of the intensity of the bands by those of parallel analysis of specific quantities of biotinilated AQP1 subjected directly to PAGE and Western blot analysis.

FIG. 6 shows that the antigen has the apparent molecular weight of the AQP1-GST fusion protein (˜55 KD) and of its dimer (apparently due to GST dimerization), and that the serum antibodies (especially those of serum 1) bound to at least a large fraction of the used protein (by comparison with the intensity of the marker AQP1 bands) thus excluding the possibility that their antigen is an impurity in the AQP1 preparation with similar MW with AQP1.

### Example 7

### Identification of AQP1 Antibody-Binding Pattern in Mouse Brain with IIF

Interestingly, in humans (but not in mice), AQP1 has similar cell type distribution with AQP4 in astrocytes (Satoh, Tabunoki et al. 2007; Moftakhar, Lynch et al. 2010), However, autoantibodies to AQP1 may not be detected with the usual IIF assay on mouse tissues used for NMO-IgG antibodies because a. this assay, in order to reduce background, usually involves treatment of the test serum with guinea pig liver powder, which incidentally contains AQP1 (Talbot, Garrett et al. 2003) and therefore any autoantibodies to AQP1 in the test serum would be eliminated and not detected; and b. even if this problem is overcome, mouse astrocytes do not express AQP1.

Elimination of the liver powder step is not preferred when using of intact sera since it would result in relatively high background. However, isolation of anti-AQP1 and anti-AQP4 antibodies by affinity chromatography may allow for omission of the liver powder step.

We isolated anti-AQP1 antibodies from sera from AQP1+ patients (and from AQP4+ and NHS, as negative controls) by affinity chromatography using immobilized AQP1 With samples purified from sera positive for AQP1 antibodies we detected a new specific pattern corresponding to AQP1 expression in the choroid plexus in the mouse brain. The corresponding samples “purified” from AQP4+ sera and from NHS did not result in significant staining. When whole untreated sera with anti-AQP1 antibodies were used (without liver powder treatment) a similar staining pattern was observed, but the background (by the use of NHS) was relatively high.

### Example 8

### Clinical/MRI and Cytological Findings in Patients Positive for Anti-AQP1 Antibodies

Most test sera were collected from patients during routine diagnosis for anti-AQP4 antibodies, with minimal clinical data available. We studied in detail the clinical data for 15 patients with only anti-AQP1 antibodies (anti-AQP1+). A retrospective analysis of MRI showed that 3 of them had NMO (longitudinally extensive transverse myelitis with optic neuritis), 7 had only longitudinally extensive transverse myelitis with brain lesions atypical for MS, one had only transverse myelitis, while 4 had MS (however with predominantly spinal cord lesions, the two which had longitudinally extensive transverse myelitis). Importantly, three of the 15 patients also had neoplasm (nephroma, non-Hodgkin lymphoma, or mammary cancer), suggesting anti-AQP1 autoantibodies as a possible novel paraneoplastic marker.

Our findings suggest the identification of a specific group of demyelinated disorders, distinct from classical MS and probably distinct from ‘classical’ NMO with only anti-AQP4 antibodies. AQP4 and AQP1 have different distribution in the CNS, with AQP4 expressed predominantly in the gray matter (brain and spinal cord) and AQP1 only in the white matter. Since patients positive only for AQP1 antibodies may have demyelination only in the white matter, we believe that the new marker, anti-AQP1 antibodies, is specific for a novel demyelinated autoimmune disorder, probably close relative to anti-AQP4 containing NMO (“classical NMO”).

In addition to the CNS, AQP1 has been also detected in the peripheral nervous system (Ma, T. H., H. W. Gao, et al. (2011) Acta Pharmacol Sin 32(6): 711-715) and in non-neuronal tissues like red blood cells, liver, kidney and lungs. Therefore, autoantibodies to AQP1 may be involved and serve as markers in relevant diseases including idiopathic autoimmune hemolytic anemia (e.g. some patients improve with cortisone or even rituximab), autoimmune chronic obstructive pulmonary disease and peripheral neuropathies.

### Example 9

### Immunoadsorption of Anti-AQP1 Antibodies from Patient Sera-Therapeutic Indications

We then tested whether the autoantigen, AQP1, when immobilized could be used for the elimination of the anti-AQP1 autoantibodies from the patient's serum. AQP1 was immobilized on ELISA plates; to the plates was added anti-AQP1 positive serum and incubated. Then the treated serum was tested by RIPA to determine whether the serum was depleted from the autoantibodies. FIG. 7 shows that the serum was practically completely depleted of all anti-AQP1 autoantibodies. This experiment suggests that it is possible to use immobilized AQP1 or its fragments and mutants, to specifically eliminate the patient's antibodies from the circulation, during ex vivo immunoadsorption procedure for therapeutic purposes.

### Example 10

### Detection of Antibodies to AQP-2, -5, -7 and -8 in Sera from Patients Suspected for Having NMO/NMOsd

We examined sera from 362 patients (described in Example 1), suspected for NMO, for the presence antibodies to AQP2, -5, -7, and -8. Antibodies to AQP5 were detected by the cell based assay procedure set out in Waters, P. et al. (2008). “Aquaporin-4 antibodies in neuromyelitis optica and longitudinally extensive transverse myelitis.” Arch Neurol 65(7): 913-919. Antibodies to AQP2, -7, and -8 were detected by plain RIPA (not two step RIPA) described in Example 1.

Table 1 shows that antibodies to most of these AQPs were detected in some test sera, though with lower frequency than anti-AQP1/4 antibodies, but not in sera from healthy controls. Most positive sera had antibodies to only one AQP (11 sera), but 5 sera had antibodies to more than one AQP: One serum was positive for three AQPs (AQPs 1, 2 and 8), two sera were positive for AQPs 1 and 8, one for AQPs 4 and 8, and one serum was positive for AQPs 1 and 5.

TABLE 1
Detection of antibodies to AQP-2, -5, -7 and -8 in sera from
patients suspected for having NMO-like symptoms

| | Detection of antibodies to: | | | |
|---|---|---|---|---|
| | AQP2 | AQP5 | AQP7 | AQ P8 |
| Tested | | | | |
| sera from: | Positive/total tested | | | |
| Patients suspected for | 3/86 | 2/22 | 4/65 | 8/138 |
| NMO-like symptoms | (3.5%) | (9%) | (6.2%) | (5.8%) |
| Healthy controls | 0/28 | 0/9 | 0/12 | 0/55 |

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry, immunology and biotechnology or related fields are intended to be within the scope of the following claims.

1. A method of diagnosis or prognosis of an autoimmune disease or cancer in a subject, the method comprising detecting aquaporin-1 (AQP1), aquaporin-2 (AQP2), aquaporin-5 (AQP5), aquaporin-7 (AQP7) and/or aquaporin-8 (AQP8) autoantibodies in a sample obtained from said subject.

2. A method according to claim 1, wherein the method comprises detecting AQP1 autoantibodies in a sample obtained from said subject.

3. A method of monitoring the progress of an autoimmune disease in a subject comprising the steps of:
(i) providing a first sample from the subject,
(ii) detecting AQP1, AQP2, AQP5, AQP7 and/or AQP8 autoantibodies in said first sample,
(iii) providing a second sample from the subject wherein said second sample is obtained from the subject after said first sample,
(iv) comparing the level of the AQP1, AQP2, AQP5, AQP7 and/or AQP8 autoantibodies in the second sample with the corresponding level of AQP1, AQP2, AQP5, AQP7 and/or AQP8 autoantibodies in the first sample, wherein an increase in level of the AQP1, AQP2, AQP5, AQP7 and/or AQP8 autoantibodies in the second sample relative to the first sample indicates disease progression.

4. A method according to claim 3 wherein the second sample is obtained from the subject at least 10, 20, 50, 100, 200 or 300 days after the first sample.

5. A method according to claim 3 wherein the method is for monitoring disease progression in response to treatment of said disease.

6. A method according to claim wherein the AQP1, AQP2, AQP5, AQP7 and/or AQP8 autoantibodies are detected by contacting said sample with the respective AQP1, AQP2, AQP5, AQP7 and/or AQP8 polypeptide or fragment thereof, or a cell or tissue comprising said polypeptide or fragment, and detecting the presence or absence of binding of said AQP1, AQP2, AQP5, AQP7 and/or AQP8 autoantibodies to said AQP1, AQP2, AQP5, AQP7 and/or AQP8 polypeptide, fragment, cell or tissue.

7. A method of detecting the presence or absence of AQP1, AQP2, AQP5, AQP7 and/or AQP8 autoantibodies in a sample obtained from a subject comprising contacting said sample with the respective AQP1, AQP2, AQP5, AQP7 and/or AQP8 polypeptide or fragment thereof, or a cell or tissue comprising said polypeptide or fragment, and detecting the presence or absence of binding of said AQP1, AQP2, AQP5, AQP7 and/or AQP8 polypeptide, fragment, cell or tissue to said AQP1, AQP2, AQP5, AQP7 and/or AQP8 autoantibodies in said sample.

8. (canceled)

9. A method according to claim 1, wherein the sample is nervous tissue, blood, blood cells, blood plasma, blood serum or cerebrospinal fluid.

10. The method according to claim 1, wherein the AQP1, AQP2, AQP5, AQP7 and/or AQP8 autoantibodies are detected by an immuoassay.

11. A method according to claim 10 wherein the immunoassay is selected from enzyme-linked immunosorbent assay (ELISA), radioimmunoassay (RIA), immunoprecipitation assay, immunofluorescence assay (IFA), indirect immunofluorescence (IIF) assay enzyme linked assay (EIA), luminescence immunoassay (LIA), Western Blot assay (WB) and cell based assay (CBA).

12. (canceled)

13. A method according to claim 1, wherein the disease is an autoimmune disease of the central nervous system (CNS).

14. A method according to claim 1, wherein the disease is associated with the formation of demyelinated lesions in the CNS.

15. A method according to claim 7, wherein the method further comprises detecting aquaporin-4 (AQP4) autoantibodies.

16. A method of treating a subject having a disease characterised by the presence of, or elevated levels of, AQP1, AQP2, AQP5, AQP7 and/or AQP8 autoantibody comprising withdrawing a body fluid containing AQP1, AQP2, AQP5, AQP7 and/or AQP8 autoantibodies; removing a substantial portion of the autoantibodies from the body fluid; and returning the body fluid to the subject.

17. A method according to claim 16 wherein the body fluid is blood.

18. A method according to claim 16 wherein said method further comprises removing a substantial portion of AQP4 autoantibodies.

19. (canceled)

20. A method according to claim 16, wherein the removing comprises contacting the body fluid with a solid support comprising an AQP1, AQP2, AQP5, AQP7 and/or AQP8 polypeptide or fragment thereof, or a cell or tissue comprising said polypeptide or fragment, wherein the AQP1, AQP2, AQP5, AQP7 and/or AQP8 polypeptide or fragment thereof is bound to the solid support.

21. The method according to claim 20, wherein the solid support comprises a membrane, a bead, porous glass, silica, a resin, or a synthetic matrix.

22-23. (canceled)

24. An immunotherapy method comprising administering an AQP1, AQP2, AQP5, AQP7 and/or AQP8 polypeptide or fragment thereof to a subject having a disease characterised by the presence of, or elevated levels of, an AQP1, AQP2, AQP5, AQP7 and/or AQP8 autoantibody, wherein the AQP1, AQP2, AQP5, AQP7 and/or AQP8 polypeptide or fragment thereof modulates the immune response against AQP1, AQP2, AQP5, AQP7 and/or AQP8.

25. The method according to claim 24, wherein the AQP1, AQP2, AQP5, AQP7 and/or AQP8 polypeptide or fragment thereof is incorporated into a a nanoparticle or a liposome.

26. A method of treating a subject having a disease characterised by the presence of, or elevated levels of, AQP1, AQP2, AQP5, AQP7 and/or AQP8 autoantibody comprising administration to said subject an agent which binds to the subject's AQP1, AQP2, AQP5, AQP7 and/or AQP8 and protects the subject's AQP1, AQP2, AQP5, AQP7 and/or AQP8 against the pathogenic AQP1, AQP2, AQP5, AQP7 and/or AQP8 autoantibodies.

27. A method according to claim 26 wherein said agent is a non-pathogenic antibody fragment.

28. (canceled)

29. A method of screening for an agent that modulates the binding of an AQP1, AQP2, AQP5, AQP7 and/or AQP8 specific antibody to the respective AQP1, AQP2, AQP5, AQP7 and/or AQP8 polypeptide or fragment thereof comprising:
(i) contacting an AQP1, AQP2, AQP5, AQP7 and/or AQP8 polypeptide or fragment thereof with the respective AQP1, AQP2, AQP5, AQP7 and/or AQP8 specific antibody in the presence and absence of said agent; and
(ii) determining whether binding of the AQP1, AQP2, AQP5, AQP7 and/or AQP8 polypeptide or fragment thereof to the respective AQP1, AQP2, AQP5, AQP7 and/or AQP8 specific antibody is modulated in the presence of said agent.

30. (canceled)


SEQUENCE LISTING (10 sequences)

SEQ ID NO: 1 - DNA, 850 bp, Homo sapiens
gtacaaaaaagcaggctccaccatggccagcgagttcaagaagaagctcttctggagggcagtggtggccgagttcctggccacgaccctctttgtcttcatcagcatcggttctgccctgggcttcaaatacccggtggggaacaaccagacgacggtccaggacaacgtgaaggtgtcgctggccttcgggctgagcatcgccacgctggcgcagagtgtgggccacatcagcggcgcccacctcaacccggctgtcacactggggctgctgctcagctgccagatcagcatcttccgtgccctcatgtacatcatcgcccagtgcgtgggggccatcgtcgccaccgccatcctctcaggcatcacctcctccctgactgggaactcgcttggccgcaatgacctggctgatggtgtgaactcgggccagggcctgggcatcgagatcatcgggaccctccagctggtgctatgcgtgctggctactaccgaccggaggcgccgtgaccttggtggctcagccccccttgccatcggcctctctgtagcccttggacacctcctggctattgactacactggctgtgggattaaccctgctcggtcctttggctccgcggtgatcacacacaacttcagcaaccactggattttctgggtggggccattcatcgggggagccctggctgtactcatctacgacttcatcctggccccacgcagcagtgacctcacagaccgcgtgaaggtgtggaccagcggccaggtggaggagtatgacctggatgccgacgacatcaactccagggtggagatgaagcccaaattggacccagctttcttgtac

SEQ ID NO: 2 - protein, 269 aa, Homo sapiens
MASEFKKKLFWRAVVAEFLATTLFVFISIGSALGFKYPVGNNQTAVQDNVKVSLAFGLSIATLAQSVGHISGAHLNPAVTLGLLLSCQISIFRALMYIIAQCVGAIVATAILSGITSSLTGNSLGRNDLADGVNSGQGLGIEIIGTLQLVLCVLATTDRRRRDLGGSAPLAIGLSVALGHLLAIDYTGCGINPARSFGSAVITHNFSNHWIFWVGPFIGGALAVLIYDFILAPRSSDLTDRVKVWTSGQVEEYDLDADDINSRVEMKPK

SEQ ID NO: 3 - DNA, 995 bp, Homo sapiens
agtgcgagagcgagtgcccggagcatcctggccctgagacagctgggccagccccgcagggctctgcagcatgtgggagctccgctccatagccttctccagggctgtgttcgcagagttcctggccacactcctcttcgtcttctttggcctcggctctgccctcaactggccacaggccctgccctctgtgctacagattgccatggcgtttggcttgggtattggcaccctggtacaggctctgggccacataagcggggcccacatcaaccctgccgtgactgtggcctgcctggtgggctgccacgtctccgttctccgagccgccttctacgtggctgcccagctgctgggggctgtggccggagccgctctgctccatgagatcacgccagcagacatccgcggggacctggctgtcaatgctgtgagtagccacaactttgctcttctctgtccccagctcagcaacagcacgacggctggccaggcggtgactgtggagctcttcctgacactgcagctggtgctctgcatcttcgcctccaccgatgagcgccgcggagagaacccgggcacccctgctctctccataggcttctccgtggccctgggccacctccttggggtaggtcatggccatgccctcctcccactgcagatccattacaccggctgctctatgaatcctgcccgctccctggctccagctgtcgtcactggcaaatttgatgaccactgggtaatggctgaatccgctcgcccccaggtcttctggatcggacccctggtgggcgccatcctgggctccctcctctacaactacgtgctgtttccgccagccaagagcctgtcggagcgcctggcagtgctgaagggcctggagccggacaccgattgggaggagcgcgaggtgcgacggcggcagtcggtggagctgcactcgccgcagagcctgccacggggtaccaaggcctgagggccgccagcgg

SEQ ID NO: 4 - protein, 271 aa, Homo sapiens
MWELRSIAFSRAVFAEFLATLLFVFFGLGSALNWPQALPSVLQIAMAFGLGIGTLVQALGHISGAHINPAVTVACLVGCHVSVLRAAFYVAAQLLGAVAGAALLHEITPADIRGDLAVNALSNSTTAGQAVTVELFLTLQLVLCIFASTDERRGENPGTPALSIGFSVALGHLLGIHYTGCSMNPARSLAPAVVTGKFDDHWVFWIGPLVGAILGSLLYNYVLFPPAKSLSERLAVLKGLEPDTDWEEREVRRRQSVELHSPQSLPRGTKA

SEQ ID NO: 5 - DNA, 848 bp, Homo sapiens
gggtccgtgggtctgtcctctgtggggtggggggcaggtcttcaaggtctgggctcagcgccctgactcgtgccctgtctccaccaggttttctgggtagggcccatcgtgggggcggtcctggctgccatcctttacttctacctgctcttccccaactccctgagcctgagtgagcgtgtggccatcatcaaaggcacgtatgagcctgacgaggactgggaggagcagcgggaagagcggaagaagaccatggagctgaccacccgctgaccagtgtcaggcaggggccagcccctcagcccctgagccaagggggaaaagaagaaaaagatcctaacacaagcttcctttttgcacaaccggtcctcttggctgaggaggaggagctggtcaccctggctgcacagttagagaggggagaaggaacccatgatgggactcctggggtaggggccaggggctggggtctgctggggacaggtctctctgggacagacctcagagattctcaatgcagtgccaagctcacaggctgcaagggccaggccagaaaagggtgggcctgcagcctgcaccccccaccttccccaacccttcctcaagagctgaagggatcccagaccctaggtgggcagaggcagaccctccccagagctccttaggaagaagacagactggttcattgaatgccgccttatttatttctggtgaggatgcatgcgtggggctgctggtctttagagtgggggctacccaataaatcactgatactcacattccgcctctgtctctcctcagagtgccttgagacactctgccccattgcctctcctctttgtcatccc

SEQ ID NO: 6 - protein, 265 aa, Homo sapiens
MKKEVCSVAFLKAVFAEFLATLIFVFFGLGSALKWPSALPTILQIALAFGLAIGTLAQALGPVSGGHINPAITLALLVGNQISLLRAFFYVAAQLVGAIAGAGILYGVAPLNARGNLAVNALNNNTTQGQAMVVELILTFQLALCIFASTDSRRTSPVGSPALSIGLSVTLGHLVGIYFTGCSMNPARSFGPAVVMNRFSPAHWVFWVGPIVGAVLAAILYFYLLFPNSLSLSERVAIIKGTYEPDEDWEEQREERKKTMELTTR

SEQ ID NO: 7 - DNA, 1258 bp, Homo sapiens
ggctctggactggggacacagggatagctgagccccagctgggggtggaagctgagccagggacagtcacggaggaacaagatcaagatgcgctgtaactgagaagcccccaaggcggaggctgagaatcagagacatttcagcagacatctacaaatctgaaagacaaaacatggttcaagcatccgggcacaggcggtccacccgtggctccaaaatggtctcctggtccgtgatagcaaagatccaggaaatactgcagaggaagatggtgcgagagttcctggccgagttcatgagcacatatgtcatgatggtattcggccttggttccgtggcccatatggttctaaataaaaaatatgggagctaccttggtgtcaacttgggttttggcttcggagtcaccatgggagtgcacgtggcaggccgcatctctggagcccacatgaacgcagctgtgacctttgctaactgtgcgctgggccgcgtgccctggaggaagtttccggtctatgtgctggggcagttcctgggctccttcctggcggctgccaccatctacagtctcttctacacggccattctccacttttcgggtggacagctgatggtgaccggtcccgtcgctacagctggcatttttgccacctaccttcctgatcacatgacattgtggcggggcttcctgaatgaggcgtggctgaccgggatgctccagctgtgtctcttcgccatcacggaccaggagaacaacccagcactgccaggaacagaggcgctggtgataggcatcctcgtggtcatcatcggggtgtcccttggcatgaacacaggatatgccatcaacccgtcccgggacctgcccccccgcatcttcaccttcattgctggttggggcaaacaggtcttcagcaatggggagaactggtggtgggtgccagtggtggcaccacttctgggtgcctatctaggtggcatcatctacctggtcttcattggctccaccatcccacgggagcccctgaaattggaggattctgtggcgtatgaagaccacgggataaccgtattgcccaagatgggatctcatgaacccacgatctctcccctcacccccgtctctgtgagccctgccaacagatcttcagtccaccctgccccacccttacatgaatccatggccctagagcacttctaagcagagattatttgtgatcccatccattccccaataaagcaaggcttgtccgacaaa

SEQ ID NO: 8 - protein, 342 aa, Homo sapiens
MVQASGHRRSTRGSKMVSWSVIAKIQEILQRKMVREFLAEFMSTYVMMVFGLGSVAHMVLNKKYGSYLGVNLGFGFGVTMGVHVAGRISGAHMNAAVTFANCALGRVPWRKFPVYVLGQFLGSFLAAATIYSLFYTAILHFSGGQLMVTGPVATAGIFATYLPDHMTLWRGFLNEAWLTGMLQLCLFAITDQENNPALPGTEALVIGILVVIIGVSLGMNTGYAINPSRDLPPRIFTFIAGWGKQVFSNGENWWWVPVVAPLLGAYLGGIIYLVFIGSTIPREPLKLEDSVAYEDHGITVLPKMGSHEPTISPLTPVSVSPANRSSVHPAPPLHESMALEHF

SEQ ID NO: 9 - DNA, 1309 bp, Homo sapiens
taggagataagagtatcttgcacagcaggtgcaggtttcccagcagctcaggcaagagtccgatgtttgtgccatctgatcctgatgtctggagagatagccatgtgtgagcctgaatttggcaatgacaaggccagggagccgagcgtgggtggcaggtggcgagtgtcctggtacgaacggtttgtgcagccatgtctggtcgaactgctgggctctgctctcttcatcttcatcgggtgcctgtcggtcattgagaatgggacggacactgggctgctgcagccggccctggcccacgggctggctttggggctcgtgattgccacgctggggaatatcagtggtggacacttcaaccctgcggtgtccctggcagccatgctgatcggaggcctcaacctggtgatgctcctcccgtactgggtctcacagctgctcggggggatgctcggggctgccttggccaaggtggtgagtcctgaggagaggttctggaatgcatctggggcggcctttgtgacagtccaggagcaggggcaggtggcaggggcgttggtggcagagatcatcctgacgacgctgctggccctggctgtatgcatgggtgccatcaatgagaagacaaagggccctctggccccgttctccatcggctttgccgtcaccgtggatatcctggctgggggccctgtgtctggaggctgcatgaatcccgcccgtgcttttggacctgcggtggtggccaaccactggaacttccactggatctactggctgggcccactcctggctggcctgcttgttggactgctcattaggtgcttcattggagatgggaagacccgcctcatcctgaaggctcggtgagcagagctcgtgggattcctgctgctccaggtgtcctcagctcacctgtcccagactgaggacaggggagttcctgcatttcctgccagggcagaggcccagaggagcgaccccctgcttccactgcttgggcctgctttctcagatagactgactgctgaggaggctctaggttcttggaattcctttgtgctcatcagagaccccagcctggggaacacgctgcccgcactgcccagagagcagtgcaaacaccacaacacgagcgtgtttcttgagaggaatgtccccgagttggacaaggaggctgtttctgcacatcagctcatttcccgcaccccatttcttgcttgattgctttgttgggggcctggccacttccttgcttctcaagctgacaattctcactttgcaataaatagtccagtgtttccttcat

SEQ ID NO: 10 - protein, 261 aa, Homo sapiens
MSGEIAMCEPEFGNDKAREPSVGGRWRVSWYERFVQPCLVELLGSALFIFIGCLSVIENGTDTGLLQPALAHGLALGLVIATLGNISGGHFNPAVSLAAMLIGGLNLVMLLPYWVSQLLGGMLGAALAKAVSPEERFWNASGAAFVTVQEQGQVAGALVAEIILTTLLALAVCMGAINEKTKGPLAPFSIGFAVTVDILAGGPVSGGCMNPARAFGPAVVANHWNFHWIYWLGPLLAGLLVGLLIRCFIGDGKTRLILKAR